(19) United States
(12) Patent Application Publication
(10) Pub. No.: US 2016/0242839 A1
(43) Pub. Date: Aug. 25, 2016

(54) BLOOD VESSEL DISSECTING DEVICE, BLOOD VESSEL DISSECTING METHOD AND BLOOD VESSEL HARVESTING METHOD

(71) Applicant: TERUMO KABUSHIKI KAISHA, Tokyo (JP)

(72) Inventors: Makoto JINNO, Tokyo (JP); Tatsunori FUJII, Ebina-city (JP)

(73) Assignee: TERUMO KABUSHIKI KAISHA, Tokyo (JP)

(21) Appl. No.: 15/047,272

(22) Filed: Feb. 18, 2016

(30) Foreign Application Priority Data
Feb. 20, 2015 (JP) 2015-032246

(51) Int. Cl. A61B 18/14

(52) U.S. Cl. CPC A61B 18/1445; A61B 2018/00601

(57) ABSTRACT

A blood vessel dissecting device includes: a guide to be inserted into a living body along a blood vessel; and a cutting device which is connectable to the guide and which is adapted to be inserted into the living body while being guided by the guide and to cut tissue surrounding the blood vessel in the direction of alignment thereof with the blood vessel. The cutting device is deformed by an external force in the living body.

### CROSS-REFERENCES TO RELATED APPLICATIONS

This application is a continuation of Japanese Application No. 2015-032246 filed on Feb. 20, 2015, the entire content of which is incorporated herein by reference.

### TECHNICAL FIELD

The present disclosure generally relates to a blood vessel dissecting device, a blood vessel dissecting method and a blood vessel harvesting method.

### BACKGROUND DISCUSSION

It is widely known to use an artery graft represented by an internal thoracic artery, a gastroepiploic artery and a radial artery or a vein graft represented by a great saphenous vein as a bypass vessel in performing vascular bypass grafting at the heart (coronary artery bypass grafting: CABG). At present, it has been reported that artery grafts (particularly, internal thoracic artery grafts) offer higher long-term patency rates than vein grafts. Thus, vein grafts are commonly said to be poor in long-term patency rate. In recent years, however, it has been reported that the long-term patency rate concerning a vein graft is enhanced when the vein graft is harvested in the state of being covered with the surrounding tissue (for example, fat, connective tissue, tissue between a skin layer and a muscle layer, tissue between a skin layer and an interosseous membrane, branch vessels, etc.) and is used as a bypass vessel while remaining covered with the tissue. An example of a device by which a vein graft can be harvested in the state of being covered with the surrounding tissue is disclosed in US Application Publication No. 2006/0276815.

In using the device disclosed in US Application Publication No. 2006/0276815, a guide wire (support member 50) is inserted into a blood vessel to be harvested as a bypass vessel, and a tubular member (portion 40) is pushed forward while being guided by the guide wire, whereby the blood vessel can be harvested in the state of being covered with the surrounding tissue. The device disclosed in US Application Publication No. 2006/0276815, however, has drawbacks in that the guide wire may damage the internal wall of the blood vessel being harvested and that the workability in blood vessel harvesting (blood vessel dissection) is poor.

### SUMMARY

The blood vessel dissecting device, blood vessel dissecting method and blood vessel harvesting method disclosed here permit dissection of a blood vessel with good workability.

In an aspect, there is provided a blood vessel dissecting device including: an elongated guide configured to be inserted into a living body along a blood vessel; a cutting device connectable to the guide and configured to be inserted into the living body and moved in the living body while connected to the elongated guide so that the cutting device is guided by the guide and cuts tissue surrounding the blood vessel in a direction of alignment with the blood vessel. The cutting device is deformable by an external force applied to the cutting device while the cutting device is in the living body.

Preferably, the blood vessel dissecting device includes two of the guides configured to be disposed to face each other, with the blood vessel interposed therebetween, in a state of being inserted in the living body, and the cutting device is connected with both of the two guides.

In the blood vessel dissecting device, the cutting device may be deformed in a direction in which the two guides are aligned, in accordance with a separated distance between the two guides.

In the blood vessel dissecting device, preferably, the cutting device is deformed so as to protrude toward an outer side of a region between the two guides.

In the blood vessel dissecting device, the cutting device may have a bendable/curvable section which is bent or curved by the external force.

In the blood vessel dissecting device, preferably, the cutting device includes: a first portion; and a second portion provided to be slidable relative to the first portion in a direction intersecting an insertion direction of the cutting device into the living body.

In the blood vessel dissecting device, the guide may have a function to be inserted between adjacent tissues different in properties so as to dissect the adjacent tissues from each other.

In the blood vessel dissecting device, preferably, the cutting device includes: a cutting section adapted to cut tissue surrounding the blood vessel; and a treating section adapted to cut and stanch a branch vessel branched from the blood vessel.

In another aspect, there is provided a blood vessel dissecting method including: inserting a guide into a living body; moving the guide along a blood vessel in the living body to dissect tissue in a direction of alignment of the blood vessel and the guide; inserting a cutting device into the living body and moving the cutting device along the blood vessel while guiding the cutting device with the guide; and cutting tissue surrounding the blood vessel in a direction of alignment of the blood vessel and the cutting device, with the cutting of the tissue being performed by the cutting device. The cutting device is deformed by an external force in the living body during the cutting of the tissue by the cutting device.

In a further aspect, there is provided a blood vessel harvesting method including: inserting a guide into a living body and moving the guide along a blood vessel in the living body to dissect tissue in a direction of alignment of the blood vessel and the guide; inserting a cutting device into the living body and moving the cutting device along the blood vessel while guiding the cutting device by the guide to cut tissue surrounding the blood vessel in a direction of alignment of the blood vessel and the cutting device; ligating the blood vessel after dissecting the tissue and after cutting the tissue surrounding the blood vessel, and then cutting the blood vessel; and harvesting the blood vessel, after ligating the blood vessel, so that the blood vessel which is harvested is covered with the tissue. During the cutting of the tissue, the cutting device is deformed by an external force in the living body.

According to the described aspects of the present disclosure, with the cutting device deformed according, for example, to the thickness of fat surrounding a blood vessel, it is possible to perform a blood vessel dissecting operation smoothly, without forcibly pressing the skin or fascia, by using the blood vessel dissecting device. Therefore, according to the described aspects of the present disclosure, a blood vessel dissecting operation can be carried out smoothly, irrespectively of the thickness of tissue such as fat, for example.

### BRIEF DESCRIPTION OF THE DRAWINGS

FIG. 1 is a plan view illustrating a blood vessel dissecting device according to a first embodiment of the present disclosure.

FIGS. 2A and 2B illustrate a connected state in which a dissecting device and a cutting device possessed by the blood vessel dissecting device according to the first embodiment are connected, wherein FIG. 2A depicts a natural state, and FIG. 2B shows a deformed state.

FIGS. 3A and 3B illustrate the dissecting device forming a part of the blood vessel dissecting device of FIG. 1, wherein FIG. 3A is a sectional view, and FIG. 3B is a sectional view taken along the section line 3B-3B of FIG. 3A.

FIGS. 4A and 4B are plan views illustrating the cutting device forming a part of the blood vessel dissecting device of FIG. 1, wherein FIG. 4A shows a natural state, and FIG. 4B depicts a deformed state.

FIGS. 5A and 5B are views for explaining a blood vessel harvesting method carried out using the blood vessel dissecting device of FIG. 1, wherein FIG. 5A is a cross-sectional view, and FIG. 5B is a longitudinal sectional view.

FIGS. 6A and 6B are views for explaining the blood vessel harvesting method carried out using the blood vessel dissecting device of FIG. 1, wherein FIG. 6A is a cross-sectional view, and FIG. 6B is a longitudinal sectional view.

FIGS. 7A and 7B are views for explaining the blood vessel harvesting method carried out using the blood vessel dissecting device of FIG. 1, wherein FIG. 7A is a cross-sectional view, and FIG. 7B is a longitudinal sectional view.

FIGS. 8A and 8B are plan views illustrating a cutting device forming a part of a blood vessel dissecting device according to a second embodiment of the present disclosure, wherein FIG. 8A depicts a natural state, and FIG. 8B shows a deformed state.

FIGS. 9A and 9B are cross-sectional views illustrating a cutting device forming a part of a blood vessel dissecting device according to a third embodiment of the present disclosure, wherein FIG. 9A shows a natural state, and FIG. 9B depicts a deformed state.

FIGS. 10A and 10B are plan views illustrating a cutting device forming a part of a blood vessel dissecting device according to a fourth embodiment of the present disclosure, wherein FIG. 10A shows a natural state, and FIG. 10B depicts a deformed state.

FIGS. 11A and 11B are sectional views illustrating a connected state in which the cutting device shown in FIGS. 10A and 10B and a dissecting device are connected, wherein FIG. 11A depicts a natural state, and FIG. 11B shows a deformed state.

FIGS. 12A and 12B are sectional views illustrating a connected state in which a dissecting device and a cutting device forming a part of a blood vessel dissecting device according to a fifth embodiment of the present disclosure are connected, wherein FIG. 12A shows a natural state, and FIG. 12B depicts a deformed state.

FIG. 13 is a plan view illustrating a cutting device forming a part of a blood vessel dissecting device according to a sixth embodiment of the present disclosure.

FIGS. 14A and 14B are sectional views illustrating a connected state in which the cutting device shown in FIG. 13 and a dissecting device are connected, wherein FIG. 14A depicts a natural state, and FIG. 14B shows a deformed state.

FIGS. 15A and 15B are views explaining a blood vessel harvesting method carried out using the blood vessel dissecting device possessing the cutting device shown in FIG. 13.

FIG. 16 is a plan view illustrating a cutting device forming a part of a blood vessel dissecting device according to a seventh embodiment of the present disclosure.

FIGS. 17A, 17B and 17C are views explaining a blood vessel harvesting method carried out using the blood vessel dissecting device possessing the cutting device shown in FIG. 16.

FIGS. 18A and 18B are views explaining a blood vessel harvesting method carried out using the blood vessel dissecting device possessing the cutting device shown in FIG. 16.

FIGS. 19A to 19C illustrate a dissecting and cutting device forming a part of a blood vessel dissecting device according to an eighth embodiment of the present disclosure, wherein FIG. 19A is a plan view, FIG. 19B is a side view, and FIG. 19C is a sectional view taken along the section line 19C-19C of FIG. 19B.

FIGS. 20A and 20B are views explaining a blood vessel harvesting method carried out using the blood vessel dissecting device possessing the dissecting and cutting device shown in FIGS. 19A to 19C.

### DETAILED DESCRIPTION

A blood vessel dissecting device, a blood vessel dissecting method, and a blood vessel harvesting method according to the described aspects of the present disclosure will be described in detail below, with reference to several embodiments illustrated in the attached drawings, which embodiments represent examples of the inventive blood vessel dissecting device, blood vessel dissecting method, and blood vessel harvesting method disclosed here.

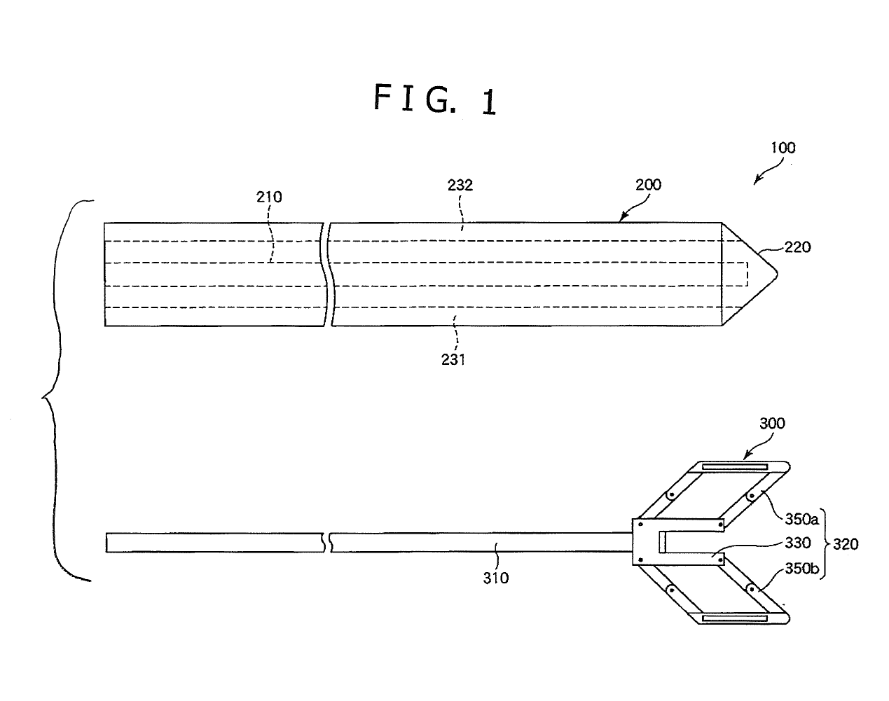

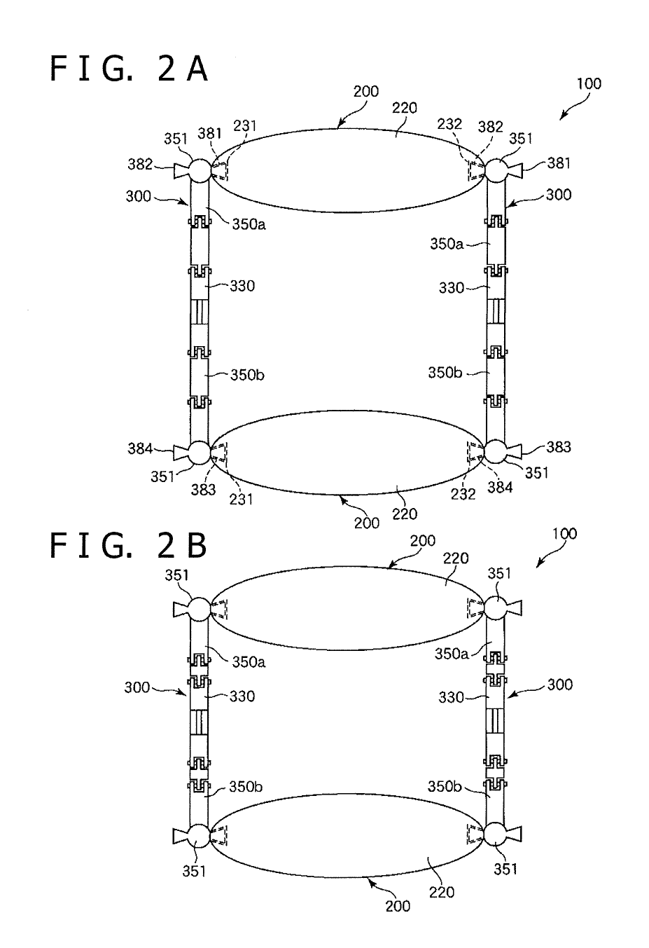

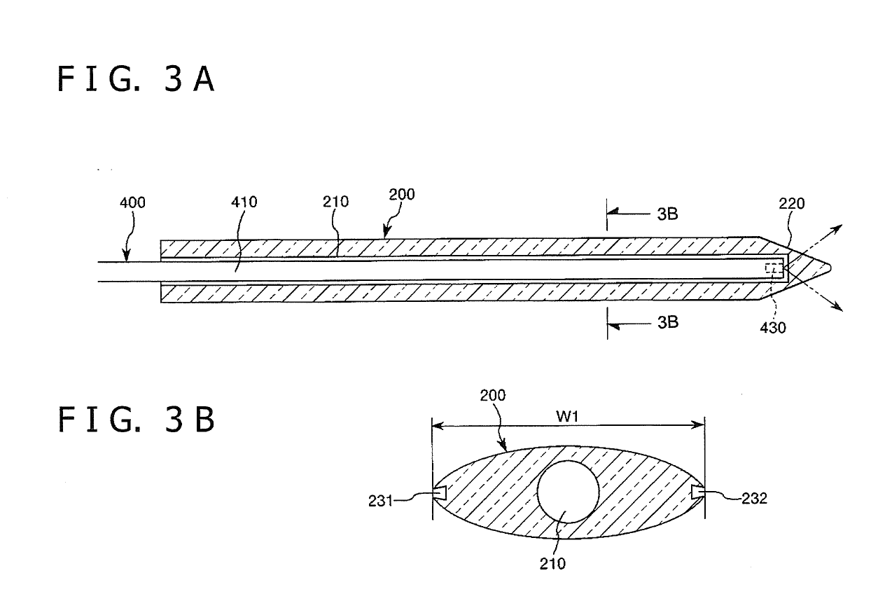

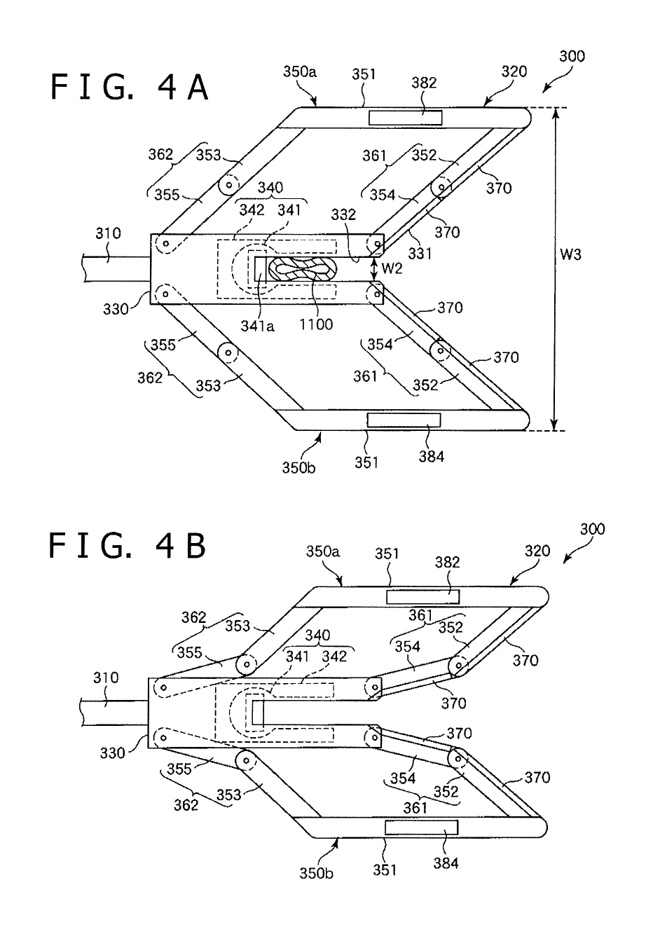

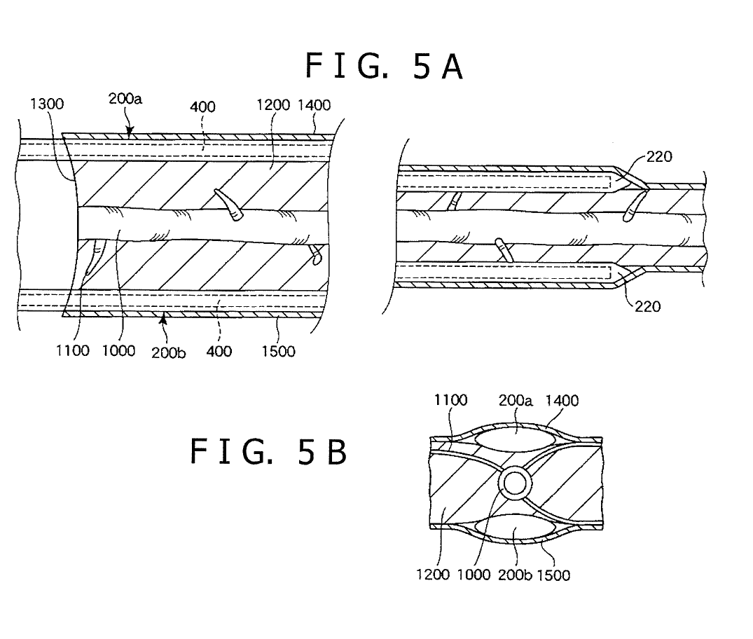

FIG. 1 is a plan view illustrating a blood vessel dissecting device according to a first embodiment of the present disclosure. FIGS. 2A and 2B illustrate a connected state in which a dissecting device and a cutting device forming a part of the blood vessel dissecting device according to the first embodiment are connected, wherein FIG. 2A depicts a natural state, and FIG. 2B shows a deformed state. FIGS. 3A and 3B illustrate the dissecting device forming a part of the blood vessel dissecting device of FIG. 1, wherein FIG. 3A is a sectional view, and FIG. 3B is a sectional view taken along the section line 3B-3B of FIG. 3A. FIGS. 4A and 4B are plan views illustrating the cutting device forming a part of the blood vessel dissecting device of FIG. 1, wherein FIG. 4A shows a natural state, and FIG. 4B depicts a deformed state. FIGS. 5A to 7B are views for explaining a blood vessel harvesting method carried out using the blood vessel dissecting device of FIG. 1, wherein FIGS. 5A, 6A and 7A are cross-sectional views, and FIGS. 5B, 6B and 7B are longitudinal sectional views. In the following detailed description, for convenience of explanation, the right side in FIG. 1 will be referred to as the “distal (side)” or distal end and the left side in FIG. 1 will be referred to as the “proximal (side) or proximal end.”

A blood vessel dissecting device 100 shown in FIG. 1 is a device used to harvest a blood vessel for use as a bypass vessel in carrying out blood vessel bypass grafting (particularly, coronary artery bypass grafting: CABG). By use of the blood vessel dissecting device 100, a blood vessel can be harvested in the state in which the blood vessel is covered with the surrounding tissue (fat, connective tissue, etc.). The blood vessel to be harvested using the blood vessel dissecting device 100 is not particularly limited insofar as it is a blood vessel that can be used as a bypass vessel. Examples of the applicable blood vessel include an internal thoracic artery, a gastroepiploic artery, a radial artery, and a great saphenous vein.

It is preferable, however, that the blood vessel to be harvested is the great saphenous vein. As aforementioned, the use of the blood vessel dissecting device 100 facilitates harvesting of a blood vessel in the state in which the blood vessel is covered with the surrounding tissue. When the great saphenous vein is harvested by using the blood vessel dissecting device 100 and is used as a bypass vessel, therefore, an enhanced long-term patency rate is obtained after the bypass grafting operation. In view of this, in the following, an example of harvesting a great saphenous vein by use of the blood vessel dissecting device 100 will be described on a representative basis.

As illustrated in FIG. 1, the blood vessel dissecting device 100 includes a dissecting device 200 (guide) and a cutting device 300. Both the dissecting device 200 and the cutting device 300 are devices which are inserted into a living body along the great saphenous vein. In addition, the blood vessel dissecting device 100 in the present embodiment includes two dissecting devices 200 and two cutting devices 300, and the dissecting devices 200 and the cutting devices 300 can be used in a connected state in which they are connected as depicted in FIG. 2A. The blood vessel dissecting device 100 shown in FIG. 2A is so configured that, when a pressure is externally exerted on the blood vessel dissecting device 100, the blood vessel dissecting device 100 can be deformed in the vertical direction as depicted in FIG. 2B. The dissecting device 200 and the cutting device 300 will now be described in detail below.

As shown in FIG. 1, the dissecting device 200 has an elongated bar-like shape (bar-shaped) extending substantially straight, and is provided at its distal end with a dissecting section 220 for dissecting tissue. In addition, as shown in FIG. 3B, the dissecting device 200 has a flat or flattened shape (vertically flattened shape) in section (transverse cross-section) as seen in FIG. 3B. The sectional shape of the dissecting device 200 is not specifically restricted; for example, the sectional shape may be a crushed-circle-like shape, such as an oblong and an ellipse, a rectangle rounded at corners, or the like.

The width (the length in the major axis direction of the cross-sectional shape) W1 of the dissecting device 200 is greater than the outside diameter of the blood vessel to be harvested (in this embodiment, the great saphenous vein). To be more specific, the width W1 is preferably about 4 mm to 4 cm greater than the outside diameter of the blood vessel to be harvested. This ensures that the possibility of contact between the cutting device 300 and the great saphenous vein can be effectively lowered at the time of inserting the cutting device 300 into the living body along the dissecting device 200, as will be explained in the “blood vessel harvesting method” described later.

In addition, the dissecting device 200 is provided, at both ends of the major axis of the cross-sectional shape thereof, with rails 231 and 232 in the form of linear stretches of recesses (or trenches) which extend in the axial direction of the dissecting device 200. The rails 231 and 232 are each a rail which is used for connection of the dissecting device 200 with the cutting device 300, and which functions as a guide section for guiding the cutting device 300. The rails 231 and 232 are not limited to the linear stretches of recesses (or trenches) but may be, for example, linear stretches of projection (or ridges or ribs), insofar as they each enable connection of the dissecting device 200 with the cutting device 300.

As shown in FIG. 3A, the dissecting device 200 is provided with an insertion hole 210 which opens at the proximal end and extends to a distal portion (the dissecting section 220). Into the insertion hole 210 is inserted an imaging device 400. The imaging device 400 is not specifically restricted. For example, the imaging device 400 in this embodiment, as depicted in FIG. 3A, includes an elongated main body section 410, and an illuminating section for emitting illumination light and an imaging section 430 for imaging the forward side of the dissecting device 200 are disposed at a distal portion of the main body section 410. The imaging section 430 includes, for example, an objective lens system disposed at the distal portion of the main body section 410 and an imaging element (e.g., solid state image sensor such as complementary metal oxide semiconductor (CMOS) image sensor or charge coupled device (CCD) sensor) disposed opposite to the objective lens system.

The dissecting section 220 is tapered in a narrowing manner toward the distal end of the dissecting device 200. More specifically, the distal end portion of the dissecting section 220 possesses a tapered roughly conical shape so that the length in the minor axis direction and the length in the major axis direction of the cross-sectional shape of the dissecting section 220 both gradually decrease toward the distal end. Such a dissecting section 220 is blunt in the thickness direction, and has such a degree of sharpness (bluntness) as to be able to dissect tissues having different properties (for example, fat and skin, fat and fascia, fat and blood vessel, fat and bone, etc.) from each other without cutting branch vessels branched from the great saphenous vein. This helps ensure that a dissecting function can be sufficiently exhibited and the branch vessels are restrained from being damaged or cut by the dissecting section 220. Accordingly, bleeding can be suppressed, and the intended procedure can be performed safely and smoothly. The shape of the dissecting section 220 is not particularly limited insofar as it enables dissection of tissues in the thickness direction (minor axis direction) of the dissecting section 220. For example, the dissecting section 220 may be in the shape of a duck bill such that the length in the minor axis direction of the cross-sectional shape of the dissecting section 220 is gradually decreased (tapered) toward the distal end and the cross-sectional shape at the distal end is a line segment along the major axis direction.

The dissecting section 220 is substantially colorless and transparent and is light-transmitting. This helps ensure that when the imaging device 400 is inserted into the insertion hole 210, the forward side of the dissecting device 200 can be observed through the dissecting section 220 by the imaging device 400. In other words, the dissecting section 220 functions as an observation section for observation of the inside of the living body (the great saphenous vein and its surroundings), in addition to the aforementioned function as the dissecting section. The dissecting section 220 is not limited to the colorless transparent property but may be colored in red, blue, green or the like, insofar as it is light-transmitting.

The cutting device 300, at the time of moving along a great saphenous vein, cuts the fat (inclusive of connective tissue) surrounding the great saphenous vein and, in addition, cuts and stanches the branch vessels branched from the great saphenous vein. As illustrated in FIG. 1, the cutting device 300 includes a main body section 320, and a bar-like operation section 310 provided on the proximal side of the main body section 320 for operation to press the main body section 320.

As shown in FIG. 4A, the main body section 320 includes a base portion 330 connected to the operation section 310, and movable portions 350a and 350b provided on both sides of the base portion 330.

The base portion 330 has an elongated plate-like shape (plate-shape) extending in the major axis direction of the cutting device 300, and has a blood vessel treating groove section 332 opening at a distal end portion of the base portion 300. The blood vessel treating groove section 332 is a groove section which has a straight shape with a substantially constant width W2 and which is for cutting and stanching a branch vessel 1100. The blood vessel treating groove section 332 is provided with a treating section 340 for cutting and stanching a branch vessel.

As depicted in FIG. 4A, the treating section 340 has a bipolar structure including a pair of electrodes 341 and 342 which generate an electric field inside the blood vessel treating section 332. The electrode 341 is provided at a proximal portion of the blood vessel treating groove section 332, whereas the electrode 342 is provided on both sides with respect to the width direction of the blood vessel treating groove section 332. With a high-frequency alternating voltage impressed between the electrodes 341 and 342, it is possible to heat and cut the branch vessel 1100 guided into the blood vessel treating groove section 332 and to stanch through thermal coagulation. A distal portion (a portion exposed to the blood vessel treating groove section 332) 341a of the electrode 341 is preferably so sharp as to be able to cut the branch vessel 1100. This helps ensure that if thermal coagulation (stanching) of the branch vessel 1100 can at least be achieved by the electric field generated between the electrodes 341 and 342, the branch vessel 1100 can be physically cut by the distal portion 341a of the electrode 341. Accordingly, the assuredness of the treatment by the treating section 340 is enhanced.

The width W2 of the blood vessel treating groove section 332 is not particularly limited but it is preferably narrower than the outside diameter of the branch vessel 1100. This helps ensure that the branch vessel 1100 can be pressed flat inside the blood vessel treating groove section 332 as depicted in FIG. 4A, and, as a result, the treatment (cutting and stanching) at the treating section 340 can be performed with enhanced reliability.

The movable portions 350a and 350b are provided on both sides of the base portion 330 and are each connected to the base portion 330. The movable portions 350a and 350b possess the same configuration except that they are symmetrical about, and on the upper and lower sides of, the base portion 330, as illustrated in FIG. 4A. Specifically, each of the movable portions 350a and 350b includes a first link 351 and bendable/curvable sections 361 and 362 for connecting the first link 351 with the base portion 330, and constitutes a parallelogram-shaped link mechanism together with the base portion 330. The movable portions 350a and 350b are each provided on substantially the same plane as the base portion 330. The components of the movable portions 350a and 350b will be described below.

The first link 351 is provided spaced from the base portion 330, and extends along the major axis direction of the base portion 330. The bendable/curvable section 361 is connected to a distal end portion of the first link 351, and the bend portion 362 is connected to a proximal portion of the first link 351.

The bendable/curvable section 361 includes a second link 352 fixed to the first link 351, and a fourth link 354 connecting the second link 352 with the base portion 330. The bend portion 362 includes a third link 353 fixed to the first link 351, and a fifth link 355 connecting the third link 353 with the base portion 330.

The second link 352 and the third link 353 extend in substantially the same direction. That is, as illustrated, the second link 352 and the third link 353 are substantially parallel.

The fourth link 354 can be rotationally moved relative to each of the second link 352 and the base portion 330. A spring member (biasing member) is provided at a connection portion between the fourth link 354 and the second link 352, and also at a connection portion between the fourth link 354 and the base portion 330, whereby the fourth link 354 is located on the same straight line as the second link 352 in a natural state (i.e., in a state in which no external force is applied, e.g., before use).

The fifth link 355 can be rotationally moved relative to each of the third link 353 and the base portion 330. A spring member (biasing member) is provided at a connection portion between the fifth link 355 and the third link 353, and also at a connection portion between the fifth link 355 and the base portion 330, whereby the fifth link 355 is located on the same straight line as the third link 353 in a natural state.

In the natural state in which no external force is exerted, each of the movable portions 350a and 350b is in the form of a parallelogram together with the base portion 330 in plan view as shown in FIG. 4A. When an external force is exerted, on the other hand, each of the movable portions 350a and 350b is deformed as depicted in FIG. 4B. The movable portions 350a, 350b in the deformed configuration possessing a different configuration/shape compared to the configuration/shape of the movable portions 350a, 350b in the natural state or non-deformed state.

Specifically, the movable portions 350a and 350b, when external forces in such directions as to bring their first links 351 closer to each other are exerted on the movable portions 350a and 350b, have their fourth links 354 and fifth links 355 tilted (falling) down to the front side, as shown in FIG. 4B, thereby being deformed so that the width W3 of the main body section 320 becomes smaller than in the natural state shown in FIG. 4A. Though not shown, the movable portions 350a and 350b, when external forces are exerted on the movable portions 350a and 350b in such directions as to move their first links 351 away from each other are, have their fourth links 354 and fifth links 355 erected to the rear side, thereby being deformed so that the width W3 of the main body section 320 becomes larger than in the natural state shown in FIG. 4A. In each of these deformed states, when the external forces are removed, the movable portions 350a and 350b are returned to their natural state shown in FIG. 4A by the restoring forces of the aforementioned spring members.

Examples of the external forces include a force which the cutting device 300 receives from tissue when the cutting device 300 is inserted into a living body, and forces which the cutting device 300 receives from the two dissecting devices 200 as the cutting device 300 moves along the separated distance between the dissecting devices 200 when the cutting device 300 is inserted into a living body in the state of being connected to the two dissecting devices 200.

In addition, as depicted in FIG. 4A, the bendable/curvable section 361 of the movable portion 350a and the bendable/curvable section 361 of the movable portion 350b form a blood vessel guide groove section (first groove section) 331. The blood vessel guide groove section 331 has a tapered shape with a width gradually decreasing toward the proximal side. The blood vessel guide groove section 331 functions as a groove section for guiding a branch vessel to the blood vessel treating groove section 332 when the cutting device 300 is pushed forward inside a living body. With the blood vessel guide groove section 331 formed in a tapered shape, the branch vessel can be guided smoothly.

The bendable/curvable sections 361 forming a part of the movable portions 350a and 350b are each provided with a cutting edge section (cutting section) 370 for cutting fat surrounding a great saphenous vein 1000. As will be explained also in the “blood vessel harvesting method” described later, the cutting edge section 370 has a function to cut fat surrounding the great saphenous vein 1000 when the cutting device 300 is pushed forward in the distal direction inside a living body. Such a cutting edge section 370 preferably has such a degree of sharpness as to be able to cut fat without cutting the branch vessels 1100. This helps ensure that the possibility of cutting of the branch vessels 1100 by the cutting edge sections 370 is lowered, so that bleeding can be restrained, and the intended procedure can be performed safely and smoothly.

In addition, the first links 351 have peripheral surfaces (side surfaces and distal surfaces) rounded so as to function as protection sections. As will be explained also in the “blood vessel harvesting method” described later, the first links 351 are moved along and between fat and skin while dissecting them from each other when the cutting device 300 is pushed forward in the distal direction inside a living body. Since the fat and the skin having different properties are easy to dissect from each other, the first links 351 can sufficiently exhibit a dissecting function to dissect the fat and the skin from each other, notwithstanding their distal portions are rounded. In addition, rounding the first links 351 can lower the possibility that branch vessels might be damaged or cut by the first links 351. Further, skin can be restrained from being damaged (cauterized) by sliding against (friction with) the first links 351.

As shown in FIG. 2A, the first links 351 are provided with connection sections 381, 382, 383 and 384 connectable with the rails 231 and 232 of the dissecting devices 200. The connection sections 381 and 382 are disposed on opposite surface sides of the first links 351. Similarly, the connection sections 383 and 384 are disposed on opposite surface sides of the first links 351. These connection sections 381 to 384 extend in the axial direction of the cutting devices 300, and include stretches of projections (or ridges or ribs) corresponding to the stretches of recesses (or trenches) of the rails 231 and 232. The cutting devices are thus connected to the dissecting devices 200 by virtue of connecting protuberances 381-384 fitted into connecting grooves 231, 232. The provision of such connection sections 381 to 384 enables the dissecting devices 200 and the cutting devices 300 to be easily connected, as shown in FIG. 2A. In addition, the provision of such connection sections 381 to 384 prevents unintended detachment of the dissecting devices 200 and the cutting devices 300 from each other, so that the intended procedure can be carried out smoothly and accurately.

A blood vessel harvesting method carried out using a blood vessel dissecting device 100 includes: a first step (blood vessel dissecting operation) of dissecting a great saphenous vein 1000 which is covered with surrounding fat 1200 by use of the blood vessel dissecting device 100; a second step of ligating the great saphenous vein 1000 and then cutting the great saphenous vein 1000; and a third step of extracting the great saphenous vein 1000 as the vein 1000 is in the state of being covered with the surrounding fat 1200 from the living body.

Here, the thickness of the fat 1200 surrounding the great saphenous vein 1000, or the separated distance from the skin 1400 to the fascia 1500, may not be constant over the entire length of the great saphenous vein 1000 but may vary. For example, there may be a part where the thickness of the fat 1200 is relatively larger as shown at the left side in FIG. 5A and a part where the thickness of the fat 1200 is relatively smaller as depicted at the right side in FIG. 5A. The blood vessel dissecting device 100 in this embodiment is particularly suitable for use in the blood vessel harvesting method in the case where the thickness of the fat 1200 is not constant. In the following, therefore, description will be made of the blood vessel harvesting method in the case where the thickness of the fat 1200 is not constant as illustrated in FIG. 5A but is decreasing toward the right side in FIG. 5A.

For convenience of explanation, in the following description of the blood vessel harvesting method, one of the two dissecting devices 200 possessed by the blood vessel dissecting device 100 will be referred to as “dissecting device 200a” and the other will be referred to “dissecting device 200b.” In the following description, one of the two cutting devices 300 will be referred to as “cutting device 300a” and the other will be referred to as “cutting device 300b.”

First, the position of the great saphenous vein 1000 to be harvested is confirmed, and a skin is incised on the basis of the position of the vein.

Next, the dissecting device 200a with the imaging device 400 inserted therein is prepared, and, while observing the inside of the living body by the imaging device 400, the dissecting device 200a is inserted via an incision 1300 into the living body along the great saphenous vein 1000 while keeping the dissecting device 200a spaced from the great saphenous vein 1000. Then, as shown in FIG. 5A, the dissecting device 200a is disposed on the upper side (the skin 1400 side) of the great saphenous vein 1000. In this case, the dissecting device 200a is so disposed that the thickness direction of the dissecting device 200a agrees substantially with the aligning direction in which the dissecting device 200a and the great saphenous vein 1000 are aligned. It is thus intended that the dissecting device 200a (and 200b) may be arranged in the vertical direction with a little position gap in the horizontal direction. The device 200a (and 200b) and the great saphenous vein 1000 can thus be positioned so that they lie in a common plane. In this operation, the dissecting device 200a is inserted between the fat 1200 and the skin 1400 (between tissues having different properties), and the skin 1400 and the fat 1200 are dissected or separated from each other in the thickness direction of the dissecting device 200 (in the aligning direction in which the dissecting device 200 and the great saphenous vein 1000 are aligned). Such an area is an area where dissection can be achieved particularly easily, so that this operation can be carried out with enhanced smoothness and accuracy.

In addition, like the dissecting device 200a, the dissecting device 200b with the imaging device 400 inserted therein is inserted into the living body. In this instance, the dissecting device 200b is inserted between the fat 1200 and the fascia 1500 (into a boundary portion between tissues having different properties), on the lower side (the fascia 1500 side (bone side)) of the great saphenous vein 1000. Such an area, also, is an area where dissection can be achieved particularly easily, so that this operation can be performed with enhanced smoothness and accuracy.

In the above-mentioned manner, the dissecting devices 200a and 200b are disposed in facing relation to each other, with the great saphenous vein 1000 interposed between the two dissecting devices 200a and 200b as illustrated in FIG. 5B.

Subsequently, the cutting device 300a is prepared, the connection section 381 of the cutting device 300a is connected to the rail 231 of the dissecting device 200a, and the connection section 383 of the cutting device 300a is connected to the rail 231 of the dissecting device 200b. By this, the cutting device 300a is connected to both of the two dissecting devices 200a and 200b. Similarly, the cutting device 300b is prepared, the connection section 382 of the cutting device 300b is connected to the rail 232 of the dissecting device 200a, and the connection section 384 of the cutting device 300b is connected to the rail 232 of the dissecting device 200b. By this, the cutting device 300b is connected to both of the two dissecting devices 200a and 200b.

Next, the cutting devices 300a and 300b are inserted into the living body while guiding the cutting devices 300a and 300b by way of the two dissecting devices 200a and 200b. In this case, the cutting devices 300a and 300b are moved forward while dissecting the skin 1400 from the fat 1200 by the first link 351 forming a part of the movable portion 350a and while dissecting the fascia 1500 from the fat 1200 by the first link 351 forming a part of the movable portion 350b. Furthermore, the cutting devices 300a and 300b each cut the fat 1200 present on a lateral side of the great saphenous vein 1000 by the cutting edge section 370 in the left-right direction (in the aligning direction in which the cutting device 300 and the great saphenous vein 1000 are aligned), and, concurrently, cut and stanch the branch vessel 1100 by the treating section 340.

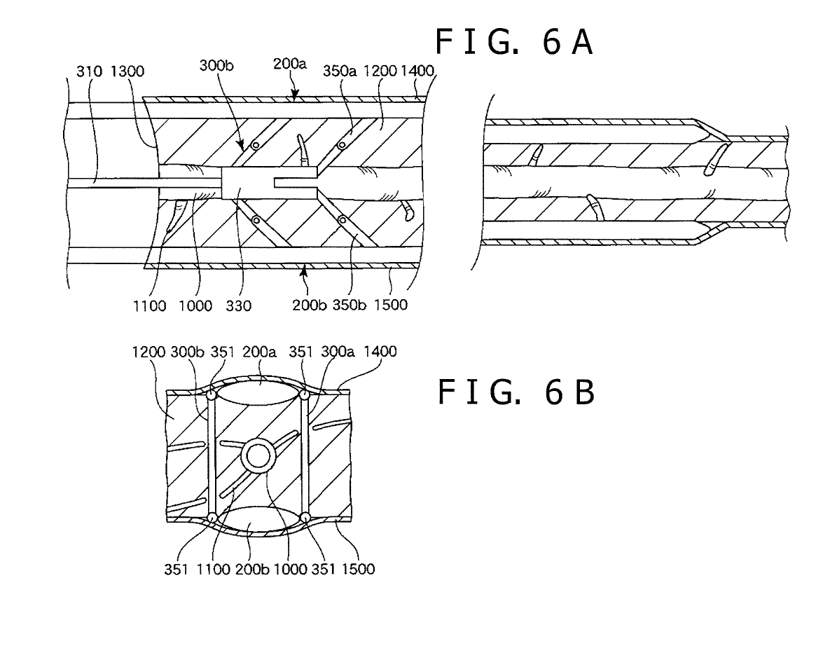

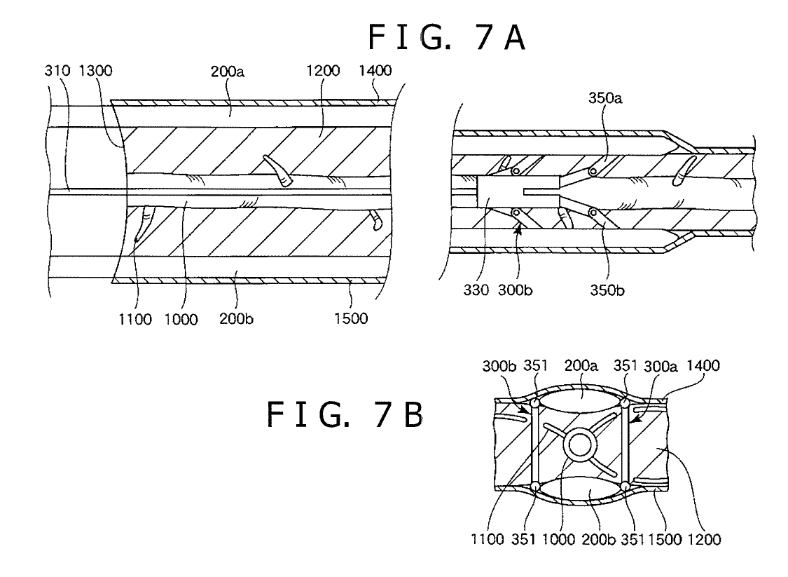

In addition, in the operation of inserting the cutting devices 300a and 300b, the cutting devices 300a and 300b are moved forward while being deformed in accordance with the separated distance between the dissecting devices 200a and 200b, in the aligning direction in which the dissecting devices 200a and 200b are aligned. Specifically, when the separated distance between the dissecting devices 200a and 200b is substantially the same as the width W3 of the main body section 320 of the cutting devices 300a and 300b, the cutting devices 300a and 300b are not deformed and are in a natural state, as illustrated in FIGS. 6A and 6B. On the other hand, when the separated distance between the dissecting devices 200a and 200b decreases, the cutting devices 300a and 300b are deformed so that the width W3 of the main body section 320 is smaller than in the natural state, following the decrease in the separated distance, as shown in FIGS. 7A and 7B. Further, though not illustrated, when the separated distance between the dissecting devices 200a and 200b increases, the cutting devices 300a and 300b are deformed so that the width W3 of the main body section 320 is greater than in the natural state, following up to the increase in the separated distance. In this way, in this operation, the cutting devices 300a and 300b are moved while being deformed in accordance with the separated distance between the dissecting devices 200a and 200b.

Where such deformable cutting devices 300a and 300b are used, the cutting devices 300a and 300b can be smoothly inserted irrespective of the thickness of the fat 1200. Therefore, the blood vessel dissecting operation can be performed smoothly and with low invasiveness, without forcibly pressing the skin 1400 or the fascia 1500.

Even when the cutting devices 300a and 300b are deformed, the cutting edge sections 370 provided at the bendable/curvable sections 361 are oriented forward, in the same manner as in the natural state. Therefore, the cutting devices 300a and 300b can, even in the deformed state, cut the fat surrounding the great saphenous vein 1000 in the same manner as when they are in the natural state.

In addition, as described above, even when the cutting devices 300a and 300b are deformed, the blood vessel guide groove sections 331 possessed by the cutting devices 300a and 300b are in the shape of gradually decreasing in width from the distal end of the main body section 320 toward the blood vessel treating groove section 332. Therefore, the cutting devices 300a and 300b can, even in the deformed state, smoothly guide the branch vessel 1100 to the blood vessel treating groove section 332 by the blood vessel guide groove section 331, and, as a result, can cut and stanch the branch vessel 1100, in the same manner as when they are in the natural state.

Since the width W1 of the dissecting devices 200a and 200b is greater than the outside diameter of the great saphenous vein 1000 as aforementioned, the cutting devices 300a and 300b can be pushed forward along the great saphenous vein 1000 while keeping the cutting devices 300a and 300b laterally spaced from the great saphenous vein 1000, as shown in FIGS. 6B and 7B. Therefore, the great saphenous vein 1000 can be prevented from being damaged during this operation. Since the first links 351 are rounded, the possibility of damaging the skin 1400 or the fascia 1500 by contact with the cutting device 300 can be lowered.

In addition, the dissecting devices 200a and 200b and the cutting devices 300a and 300b are separate bodies from each other, and are freely detachable from each other, as described above. Therefore, it is possible to insert the dissecting devices 200a and 200b and then insert the cutting devices 300a and 300b while guiding the cutting devices 300a and 300b with the dissecting devices 200a and 200b. Therefore, the risk of cutting a part that is not to be cut, by the cutting device 300a or 300b by mistake, can be avoided.

By the operations as above, the fat 1200 surrounding the great saphenous vein 1000 is dissected over the entire perimeter thereof, and the great saphenous vein 1000 is dissected in the state in which the great saphenous vein 1000 is covered with the surrounding fat 1200. The thickness of the fat 1200 dissected together with the great saphenous vein 1000 and surrounding the great saphenous vein 1000 is not particularly limited. It is preferable, however, that the thickness is about 0.1 mm to 10 mm, more preferably about 1 mm to 8 mm, and further preferably about 3 mm to 5 mm.

Next, the two cutting devices 300a and 300b and the two dissecting devices 200a and 200b are drawn out, and both ends of that part of the great saphenous vein 1000 which is to be harvested are ligated and then cut.

Subsequently, the great saphenous vein 1000 is extracted, in the state in which the great saphenous vein 1000 is covered with the surrounding fat 1200, to the outside of the living body via the incision 1300.

By the steps (first to third steps) as described above, the great saphenous vein 1000 is harvested in a state in which the great saphenous vein 1000 is covered with the surrounding fat 1200. In such a method, while using the dissecting device 200 for treating a part which is easy to dissect so as to reduce such damages as bleeding and while using the cutting device 300 for treating the fat which is difficult to dissect, the great saphenous vein 1000 can be harvested smoothly and with low invasion. In addition, since the first step can be carried out without cutting the great saphenous vein 1000, blood can be kept flowing through the great saphenous vein 1000 for a time as long as possible. Accordingly, the great saphenous vein 1000 is placed in an ischemic state for a shortened period of time, so that the great saphenous vein 1000 can be harvested with less damage.

Here, it is possible that a great saphenous vein 1000 covered with fat 1200 constitutes a bypass vessel having a superior long-term patency rate, as compared with a great saphenous vein 1000 not covered with fat 1200. The reason is considered as follows. While the great saphenous vein 1000 is used as an artery bypass vessel, arteries are considered to be higher than veins in the blood pressure (the internal pressure exerted on the vein by blood). When a great saphenous vein in an exposed state of being not covered with tissue is used as a bypass vessel, therefore, the great saphenous vein cannot endure the blood pressure and is therefore expanded by the blood pressure, possibly resulting in lowered blood flow. In addition, thickening of blood vessel wall occurs in the process of remodeling (structural alteration) or in the process of recovery from damage to tissue. Such thickening of blood vessel wall is considered to influence the development of arterial sclerosis. From such a cause, the use of a great saphenous vein in the exposed state of being not covered with tissue as a bypass vessel is considered to lead, in the long run, to vascular occlusion.

On the other hand, where the great saphenous vein 1000 is covered with fat 1200, there is a possibility that expansion of the great saphenous vein 1000 is restrained by the fat 1200, and bending and the like of the great saphenous vein 1000 are also restrained. It is considered, therefore, that the lowering in blood flow as above-mentioned can be inhibited. In addition, it is possible that the covering with the fat 1200 reduces damages to the great saphenous vein 1000, specifically, damages to endotheliocytes, smooth muscles, nutrient vessels (capillary plexus), etc. It is considered, therefore, that the aforementioned thickening of blood vessel walls can be restrained. For these reasons, the use of the great saphenous vein 1000 covered with the fat 1200 as a bypass vessel enables an excellent long-term patency rate. Especially, in this embodiment, nutrient vessels are left at the blood vessel walls of the great saphenous vein 1000 and in the fat 1200. For this reason, nutrients are supplied to the great saphenous vein 1000 serving as the bypass vessel, even after the bypass grafting. This is considered to be the reason why the aforementioned effect is enhanced.

While this embodiment has been described, the configuration of the blood vessel dissecting device 100 is not limited to the one in this embodiment. For example, the rails 231 and 232 may be omitted from the dissecting device 200, and the connection sections 381 to 384 may be omitted from the cutting device 300. In this case, for example, it may be sufficient to insert the cutting device 300 into a living body along the dissecting device 200 which is inserted into the living body earlier. That is, the cutting device(s) 300 can be inserted at positions similar to that shown in FIG. 6B, but not connected to the dissecting devices 200.

The blood vessel harvesting method is not limited to the procedure adopted in this embodiment. For example, the order in which the two cutting devices 300a and 300b are inserted is not specifically restricted. The fat 1200 present on a lateral side of the great saphenous vein 1000 may be cut to the left and right by the cutting device 300a on one side, and, after this cutting is finished, the fat 1200 present on a lateral side of the great saphenous vein 1000 may be cut to the left and right by the cutting device 300b on the other side.

While two dissecting devices 200 and two cutting devices 300 are used in this embodiment, it is sufficient for the blood vessel dissecting device 100 to have at least one dissecting device 200 and at least one cutting device 300. For instance, where the blood vessel dissecting device 100 has two dissecting devices 200 and one cutting device 300, a method may be adopted in which, first, the two dissecting devices 200 are disposed on the upper and lower sides of a great saphenous vein 1000, then the one cutting device 300 is disposed for example on one lateral side of the great saphenous vein 1000, and fat on the one lateral side of the great saphenous vein 1000 is cut, after which the cutting device 300 is drawn out of the living body. Then, the cutting device 300 thus drawn out is disposed for example on the other lateral side of the great saphenous vein 1000, and fat on the other lateral side of the great saphenous vein 1000 is cut.

While the dissecting device 200 is inserted between the fat 1200 and the skin 1400 and between the fat 1200 and the fascia 1500 in this embodiment, the insertion position of the dissecting device 200 is not particularly limited. For instance, the dissecting device 200 may be inserted between tissues having different properties, such as between the fat 1200 and a blood vessel (other than the great saphenous vein 1000), between the fat 1200 and a bone, between the fascia 1500 and a bone, or the like. Further, the insertion between tissues having different properties (insertion into the boundary between tissues having different properties, insertion into tissue between tissues having different properties, or the like) is not restrictive; for example, the dissecting device 200 may be inserted into the fat 1200, thereby dissecting the fat 1200.

While fat is cut by the cutting device 300 in this embodiment, the tissue to be cut by the cutting device 300 is not restricted to the fat. For instance, tissue between a skin-fat boundary and a fat-muscle boundary, tissue between a skin-fat boundary and a fat-interosseous membrane boundary, connective tissue, tissue between a skin layer and a muscle layer, tissue between a skin layer and an interosseous membrane, branch vessels, and the like may also be cut by the cutting device.

While the dissecting device 200 is disposed spaced from the great saphenous vein 1000 so as not to contact the great saphenous vein 1000 in this embodiment, the dissecting device 200 may be disposed in contact with the great saphenous vein 1000. In other words, the dissecting device 200 may be inserted between the great saphenous vein 1000 and the fat 1200.

While the cutting device 300 is guided into a living body by the dissecting device 200 in this embodiment, the member for guiding the cutting device 300 is not limited to the dissecting device 200. The member for guiding the cutting device 300 may not have a function to dissect tissues having different properties, insofar as the member can guide the cutting device 300 into the living body. In this case, the dissecting device 200 may be omitted, as required.

While the movable portions 350a and 350b possessed by the cutting device 300 are provided at a distal portion of the cutting device 300 in this embodiment, the movable portions 350a and 350b may be provided over substantially the entire length of the cutting device 300.

The blood vessel dissecting device 100 may have a fixation mechanism for maintaining a deformed state of the movable portions 350a and 350b possessed by the cutting device 300 when the movable portions 350a and 350b have been deformed. Where the blood vessel dissecting device 100 has the fixation mechanism, it is possible, for example, to preliminarily fix the width of the cutting device 300 according to the thickness of the fat 1200 by the fixation mechanism and then to perform a blood vessel dissecting operation. Where the fixation mechanism is thus provided for the deformable cutting device 300, a cutting device 300 having a suitable width according to the thickness of the fat 1200 can be easily prepared. As a result, the need to separately prepare a new blood vessel dissecting device 100 according to the thickness of the fat 1200 can be eliminated.

While the movable portions 350a and 350b are the same in configuration in this embodiment, the movable portions 350a and 350b may have different configurations, or either one of them may be omitted. While the cutting device 300 has two movable portions 350a and 350b in this embodiment, the cutting device 300 may be configured by use of three or more movable portions.

In this embodiment, the movable portions 350a and 350b are deformable both in such a direction that the width W3 of the main body section 320 becomes smaller than in the natural state and in such a direction that the width W3 becomes greater than in the natural state. However, the movable portions 350a and 350b may be configured to be deformable in only either one of the just-mentioned directions. For instance, the movable portions 350a and 350b may be configured to be deformable only in such a direction that the width W3 of the main body section 320 becomes smaller than in the natural state, or may be configured to be deformable only in such a direction that the width W3 of the main body section 320 becomes greater than in the natural state.

While the length of the second link 352 and the length of the third link 353 are substantially the same as the length of the fourth link 354 and the length of the fifth link 355 respectively in this embodiment, the corresponding lengths may not necessarily be the same or comparable but may be different. Thus, the length of the second link 352 and the length of the third link 353 may be shorter, or may be longer, than the length of the fourth link 354 and the length of the fifth link 355, respectively. In addition, for example, the second link 352 and the third link 353 may be omitted. Where the second link 352 and the third link 353 are omitted, it is sufficient that the fourth link 354 and the fifth link 355 can be rotationally moved in relation to the first link 351 and the base portion 330.

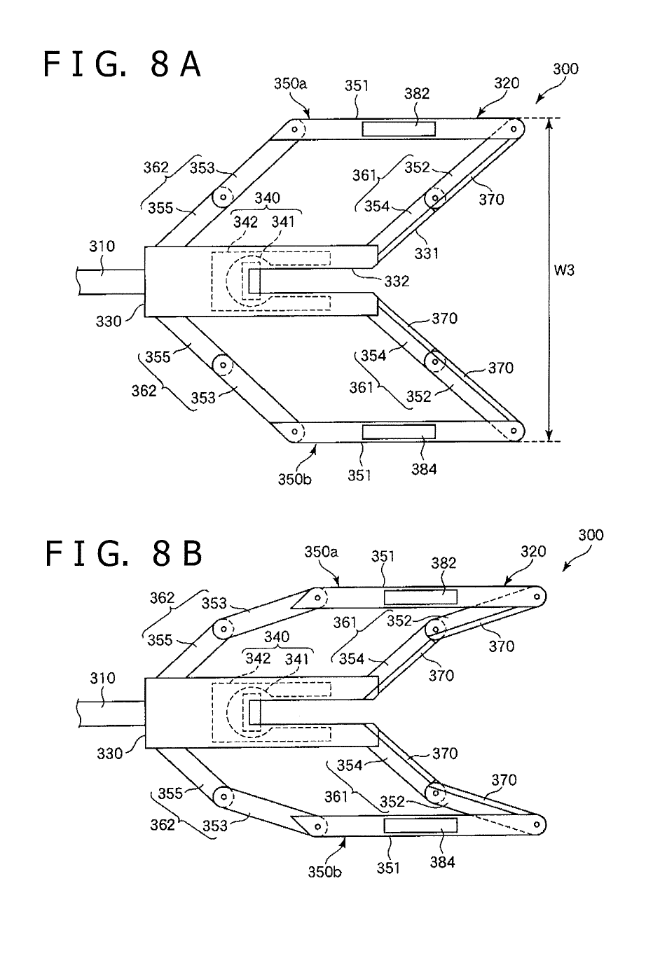

FIGS. 8A and 8B are plan views illustrating a cutting device forming part of a blood vessel dissecting device according to a second embodiment of the present disclosure, wherein FIG. 8A depicts a natural state, and FIG. 8B shows a deformed state.

Referring to these figures, the second embodiment will be described below. Features associated with this second embodiment that are similar to those in the first embodiment are identified by common reference numbers. The following detailed description will primarily focus on differences between this second embodiment and the first embodiment described above, and a detailed description of features already described above will not be repeated.

This second embodiment is the same as the first embodiment above, except for differences in the configuration of moving portions forming a part of the cutting device.

In a cutting device 300 in this embodiment, as illustrated in FIGS. 8A and 8B, the second links 352 and the third links 353 forming a part of the movable portions 350a and 350b are rotationally movable in relation to first links 351. On the other hand, the fourth links 354 and the fifth links 355 are fixed to a base portion 330.

Like in the first embodiment above, the thus configured movable portions 350a and 350b, when external forces are exerted on the movable portions 350a and 350b in such a direction that their first links 351 are brought closer to each other, are deformed so that the width W3 of a main body section 320 becomes smaller than in the natural state shown in FIG. 8A, as depicted in FIG. 8B. In addition, though not illustrated, the movable portions 350a and 350b, when external forces are exerted on the movable portions 350a and 350b in such a direction that their first links 351 are spaced away from each other, are deformed so that the width W3 of the main body section 320 becomes greater than in the natural state shown in FIG. 8A.

By the second embodiment as above, also, the same or equivalent effects to those of the aforementioned first embodiment can be produced.

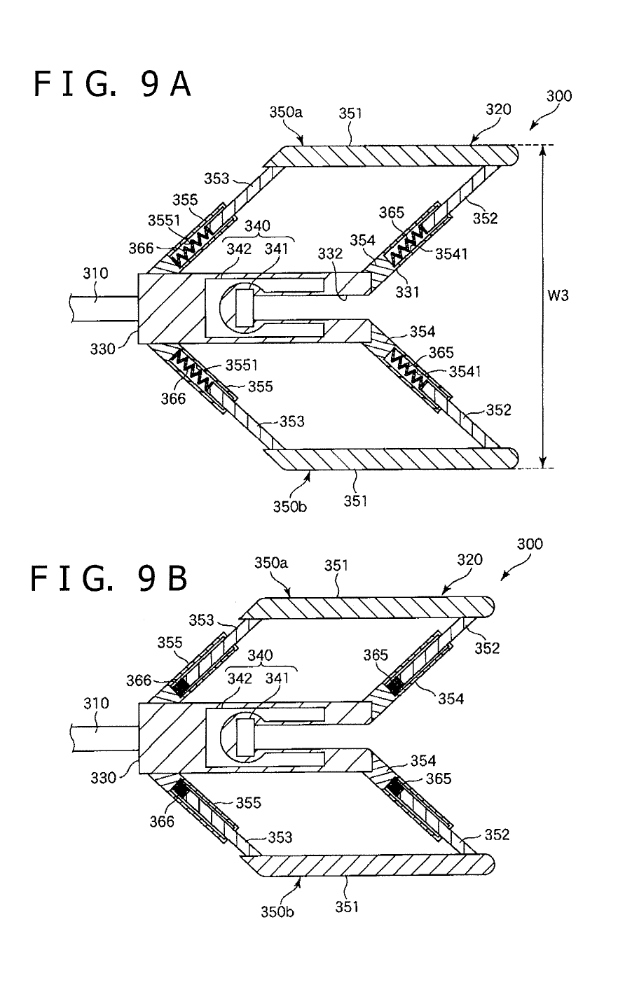

FIGS. 9A and 9B are cross-sectional views illustrating a cutting device forming a part of a blood vessel dissecting device according to a third embodiment of the present disclosure, wherein FIG. 9A shows a natural state, and FIG. 9B depicts a deformed state.

Referring to these figures, the third embodiment will be described below. Features associated with this third embodiment that are similar to those in the embodiments described above are identified by common reference numbers. The following detailed description will focus primarily on differences between this third embodiment and the embodiments described above, and a detailed description of features already described above will not be repeated.

This third embodiment is the same as the embodiments described above, except mainly for differences in the configuration of movable portions forming a part of a cutting device.

In a cutting device 300 in this embodiment, as illustrated in FIGS. 9A and 9B, fourth links 354 and fifth links 355 are fixed to a base portion 330. In addition, a second link (second portion) 352 forming a part of each of the movable portions 350a and 350b is slidable relative to a fourth link (first portion) 354 in an axial direction thereof, and a third link (second portion) 353 forming a part of each of the movable portions 350a and 350b is slidable relative to a fifth link (first portion) 355 in an axial direction thereof.

Specifically, as depicted in FIG. 9A, each fourth link 354 is provided therein with a hole 3541 which opens to the second link 352 side and extends to the base portion 330 side, and the second link 352 is slidably disposed in the hole 3541. In addition, a spring member 365 is provided in the hole 3541. The spring member 365 has its one end fixed to the fourth link 354, and has its other end fixed to the second link 352.

Similarly, each fifth link 355 is provided therein with a hole 3551 which opens to the third link 353 side and extends to the base portion 330 side, and the third link 353 is slidably disposed in the hole 3551. A spring member 366 is provided in the hole 3551. The spring member 366 has its one end fixed to the fifth link 355, and has its other end fixed to the third link 353.

The thus configured movable portions 350a and 350b, when external forces are exerted on the movable portions 350a and 350b in such a direction that their first links 351 are brought closer to each other, have their second links 352 and third links 353 slid toward a base portion 330 (in directions intersecting the direction of insertion into the living body), as depicted in FIG. 9B. Consequently, the movable portions 305a and 350b are deformed so that the width W3 of a main body section 320 becomes smaller than in the natural state shown in FIG. 9A, as depicted in FIG. 9B. Though not illustrated, the movable portions 350a and 350b, when external forces are exerted on the movable portions 350a and 350b in such a direction that their first links 351 are spaced away from each other, have their second links 352 and third links 353 slid in the direction for spacing away from the base portion 330. As a result, the movable portions 350a and 350b are deformed so that the width W3 of the main body section 320 becomes greater than in the natural state shown in FIG. 9A.

By the third embodiment as above, also, the same or equivalent effects to those of the aforementioned embodiments can be produced.

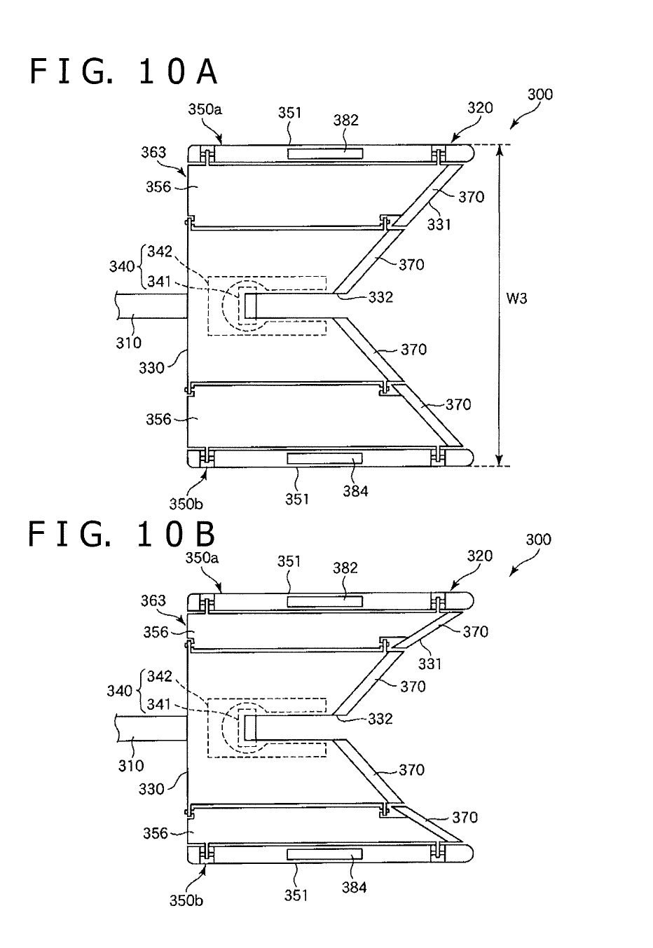

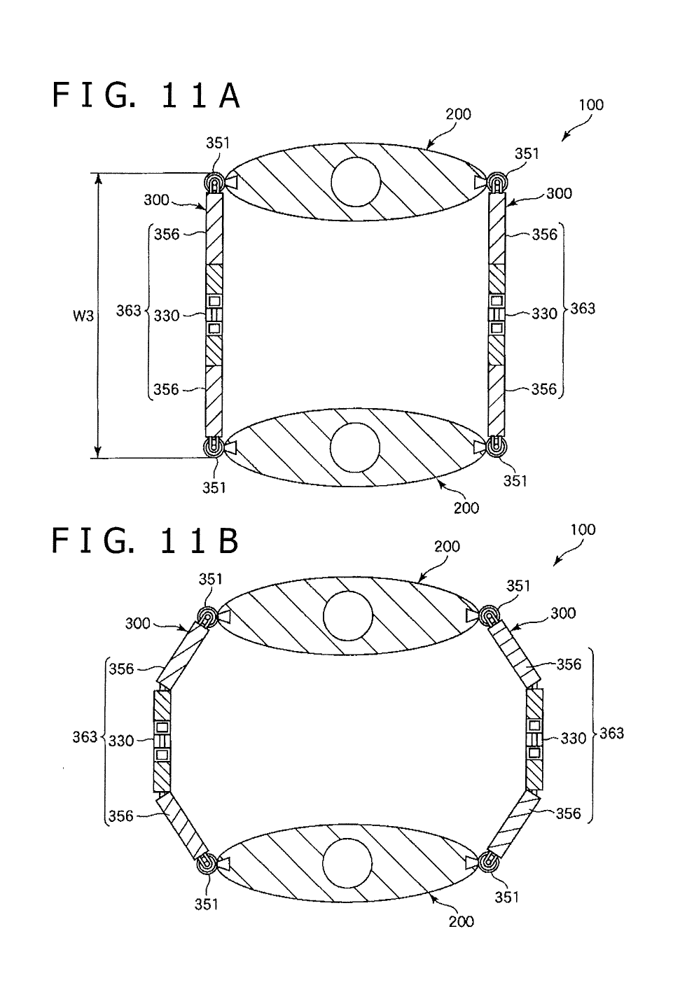

FIGS. 10A and 10B are plan views illustrating a cutting device forming a part of a blood vessel dissecting device according to a fourth embodiment of the present disclosure, wherein FIG. 10A depicts a natural state, and FIG. 10B shows a deformed state. FIGS. 11A and 11B are sectional views illustrating a connected state in which the cutting devices shown in FIGS. 10A and 10B and dissecting devices are connected, wherein FIG. 11A shows a natural state, and FIG. 11B depicts a deformed state.

Referring to these figures, the fourth embodiment will be described below. Features associated with this fourth embodiment that are similar to those in the embodiments described above are identified by common reference numbers. The following detailed description will primarily focus on differences between this fourth embodiment and the embodiments described above, and a detailed description of features already described above will not be repeated.

This fourth embodiment is the same as the aforementioned embodiments, except for differences in the configuration of cutting device.

A main body section 320 of each cutting device 300 in this embodiment, as shown in FIG. 10A, includes first links 351, and a bendable/curvable section 363 including a base portion 330 and plate members (rotary movement portions) 356.

Each plate member 356 has an elongated plate-like shape (plate-shaped) along the first link 351, and is provided between the first link 351 and the base portion 330. The plate member 356 is connected to the first link 351 and the base portion 330 so that the plate member 356 can be rotationally moved relative to the first link 351 and to the base portion 330. In addition, a spring member (biasing member) is provided at a connection portion between the plate member 356 and the first link 351 and also at a connection portion between the plate member 356 and the base portion 330. By this, the plate member 356 is located on the same plane as the base portion 330, in the natural state.

In addition, distal portions of the plate members 356 and a distal portion of the base portion 330 form a blood vessel guide groove section 331. A cutting edge section (cutting section) 370 is provided at each of the distal portions of the plate members 356 and the distal portion of the base portion 330.

The thus configured main body section 320, when external forces are exerted main body section 320 in such a direction that its first links 351 are brought closer to each other, have the plate members 356 tilted in the manner of bending at the connection portion between the plate member 356 and the base portion 330 so that the plate members 356 are oriented in an angled manner (not parallel) to the base portion 330. As a result, the main body section 320 is deformed (i.e., does not lie in a common plane) so that the width W3 of the main body section 320 (the separated distance between the first links 351) becomes smaller than in the natural state.

In this embodiment, the aforementioned spring member provided at the connection portion between the plate member 356 and the first link 351 functions as a rotary movement restriction unit for biasing the plate member 356 so as to rotationally move the plate member 356 to one side, with its position in the natural state as a reference point. Therefore, the two cutting devices 300 forming a part of the blood vessel dissecting device 100 in their state in connection with the dissecting devices 200, when external forces are exerted on the cutting devices in such a direction that their first link 351 are brought closer to each other, are deformed so as to protrude toward the outer side of the region between the two dissecting devices 200 (toward the side opposite from a great saphenous vein 1000) as depicted in FIG. 11B.

By the fourth embodiment as above, also, the same or equivalent effects to those of the aforementioned embodiments can be produced.

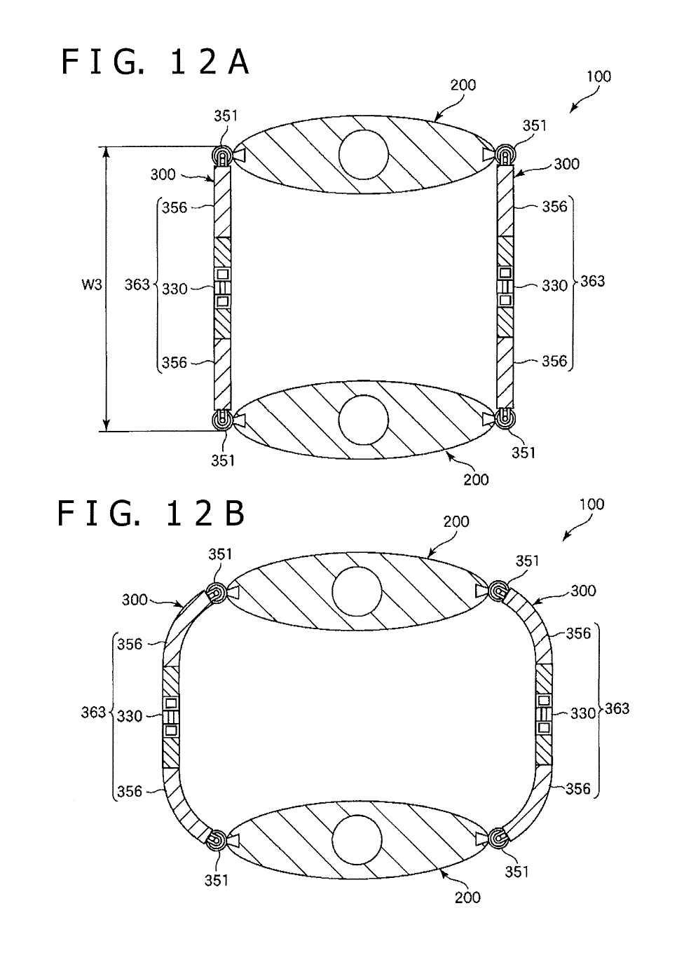

FIGS. 12A and 12B are sectional views illustrating a connected state in which dissecting devices and cutting devices forming a part of a blood vessel dissecting device according to a fifth embodiment of the present disclosure are connected, wherein FIG. 12A depicts a natural state, and FIG. 12B shows a deformed state.

Referring to these figures, the fifth embodiment will be described below. Features associated with this fifth embodiment that are similar to those in the embodiments described above are identified by common reference numbers. The following detailed description will primarily focus on differences between this fifth embodiment and the embodiments described above, and a detailed description of features already described above will not be repeated.

This fifth embodiment is the same as the aforementioned embodiments, except mainly for differences in the configuration of dissecting device.

Plate members (rotary movement portions) 356 forming a part of a bendable/curvable section 363 of a cutting device 300 in this embodiment is flexible. In addition, plate members 356 are each fixed to a base portion 330, and are each connected to a first link 351 in a rotationally movable manner.

A main body section 320 having such plate members 356, when external forces are exerted on the main body section 320 in such a direction that the first links 351 are brought closer to each other, have the plate members 356 bent, whereby the main body section 320 is deformed so that the width W3 of the main body section 320 (the separated distance between the first links 351) becomes smaller than in the natural state. Such a deformation of the main body sections 320 helps ensure that the two cutting devices 300 forming a part of the blood vessel dissecting device 100, in the connected state, when external forces are exerted on the main body section 320 in such a direction that the first links 351 are brought closer to each other, are deformed so as to protrude toward the outer sides of the region between the two dissecting devices 200, as depicted in FIG. 12B.

By the fifth embodiment as above, also, the same or equivalent effects to those of the aforementioned embodiments can be produced.

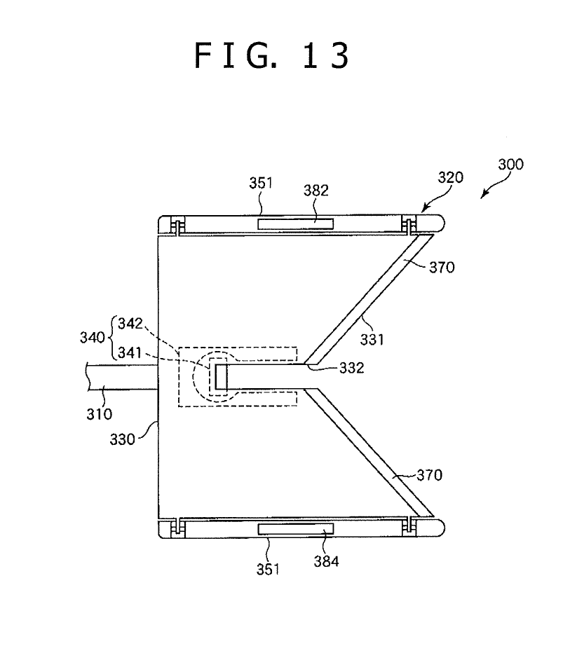

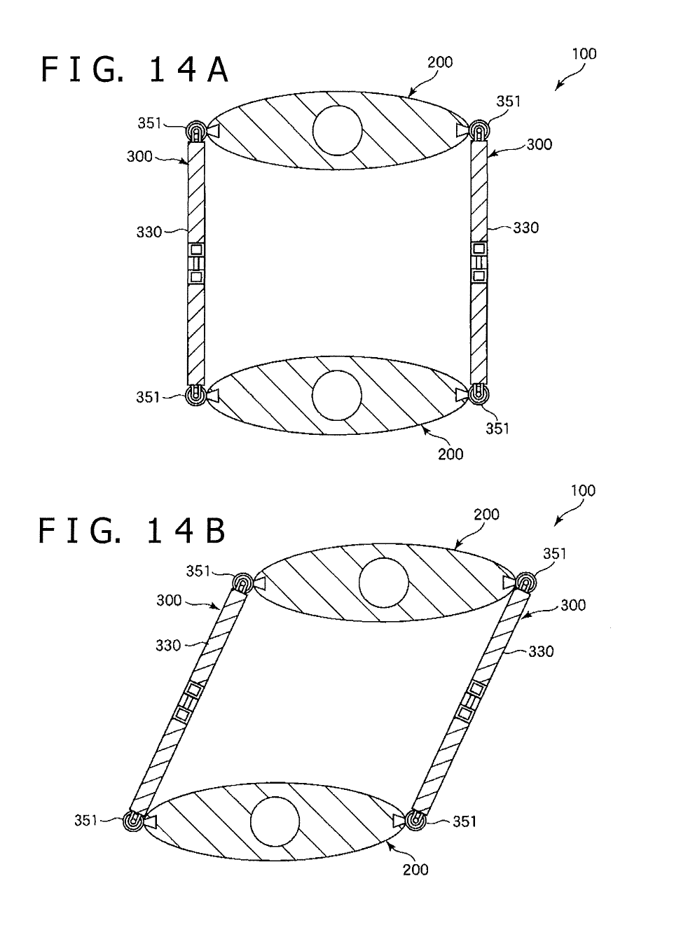

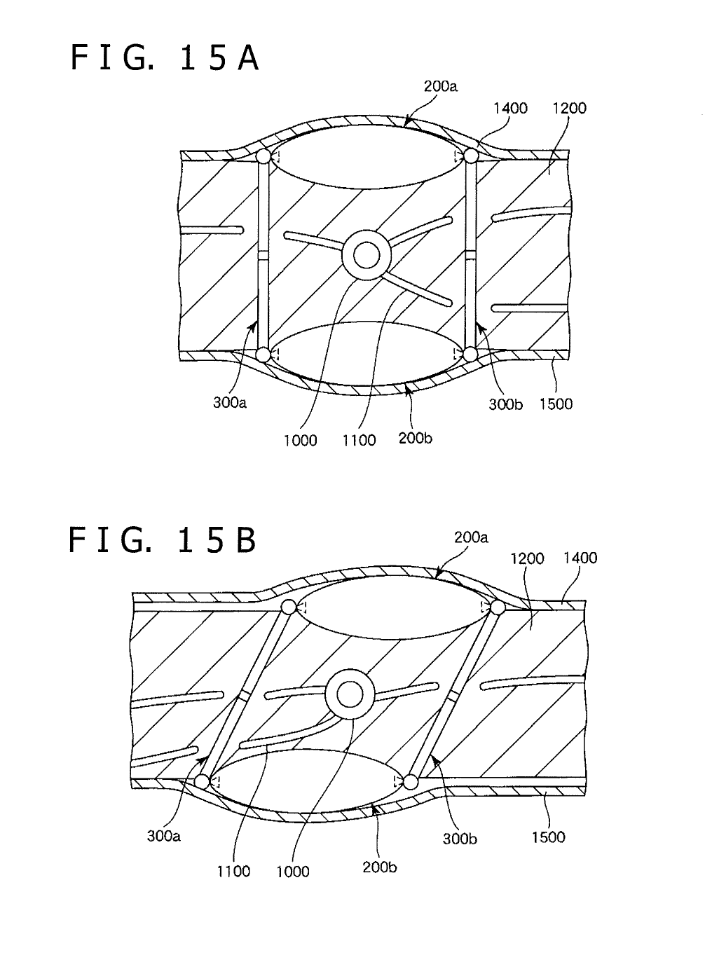

FIG. 13 is a plan view illustrating a cutting device forming a part of a blood vessel dissecting device according to a sixth embodiment of the present disclosure. FIGS. 14A and 14B are sectional views illustrating a connected state in which the cutting devices shown in FIG. 13 and dissecting devices are connected, wherein FIG. 14A depicts a natural state, and FIG. 14B shows a deformed state. FIGS. 15A and 15B are views for explaining a blood vessel harvesting method carried out using the blood vessel dissecting device forming a part of the cutting device shown in FIG. 13.

Referring to these figures, the sixth embodiment will be described below. Features associated with this sixth embodiment that are similar to those in the embodiments described above are identified by common reference numbers. The following detailed description primarily focuses on differences between this sixth embodiment and the embodiments described above, and a detailed description of features already described above of will not be repeated.

This sixth embodiment is the same as the aforementioned embodiments, except mainly for differences in the configuration of cutting device.

A main body section 320 forming a part of a cutting device 300 in this embodiment, as illustrated in FIG. 13, includes two first links 351, and a flat plate-shaped base portion (rotary movement portion) 330 connected to these first links 351.

The base portion 330 includes: a blood vessel guide groove section 331 which opens to a distal portion and has a tapered shape with a width gradually decreasing toward the proximal side; and a blood vessel treating groove section 332 located on the proximal side of the blood vessel guide groove section 331. In addition, the base portion 330 is provided at its distal portion with cutting edge sections 370 along the blood vessel guide groove section 331.

The base portion 330 as above is rotationally movable in relation to each of the first links 351. A spring member (biasing member) is provided at each connection portion between the base portion 330 and each first link 351.

A blood vessel dissecting device 100 that includes the cutting devices 300 configured as above, when external forces are not exerted on the cutting devices in a connected state, have two dissecting devices 200 overlapping each other in plan view as viewed in the thickness direction of the dissecting devices 200, as shown in FIG. 14A. On the other hand, when external forces are exerted in such a direction that the two dissecting devices 200 are brought closer to each other, the blood vessel dissecting device 100 has the two cutting devices 300 tilted in the same direction, thereby being deformed so that the two dissecting devices 200 are positionally deviated from each other in plan view.

In a first step, the two cutting devices 300a and 300b are inserted into a living body. During this insertion, as the separated distance between the dissecting devices 200a and 200b is decreased, the cutting devices 300a and 300b are displaced so as to be tilted relative to the dissecting devices 200a and 200b, as depicted in FIG. 15B from the natural state shown in FIG. 15A. Under the influence of this tilting (inclination), the dissecting devices 200a and 200b are moved sideways. In this instance, the dissecting device 200a is moved toward the right side in FIG. 15A while dissecting the skin 1400 from the fat 1200, whereas the dissecting device 200b is moved toward the left side in FIG. 15A while dissecting the fascia 1500 from the fat 1200. In other words, the dissecting devices 200a and 200b are moved in opposite directions. In this way, the blood vessel dissecting device 100 in this embodiment changes in posture according to the thickness of the fat 1200.

By use of the blood vessel dissecting device 100 whose posture can be changed, the cutting devices 300a and 300b can be smoothly inserted, irrespective of the thickness of the fat 1200. Therefore, the blood vessel dissecting operation can be carried out smoothly and with less invasion, without forcibly pressing the skin 1400 or the fascia 1500.

By the sixth embodiment as above, also, the same or equivalent effects to those of the aforementioned embodiments can be produced.

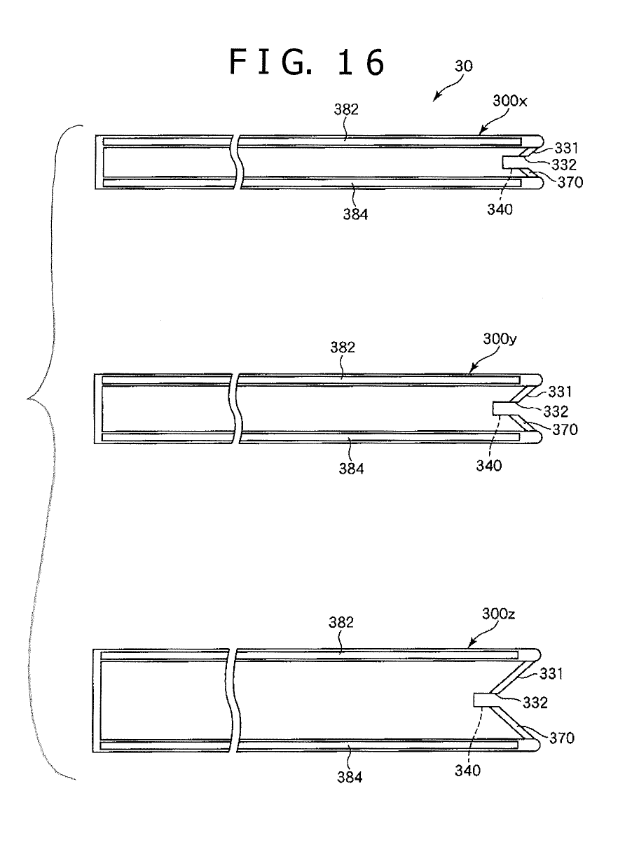

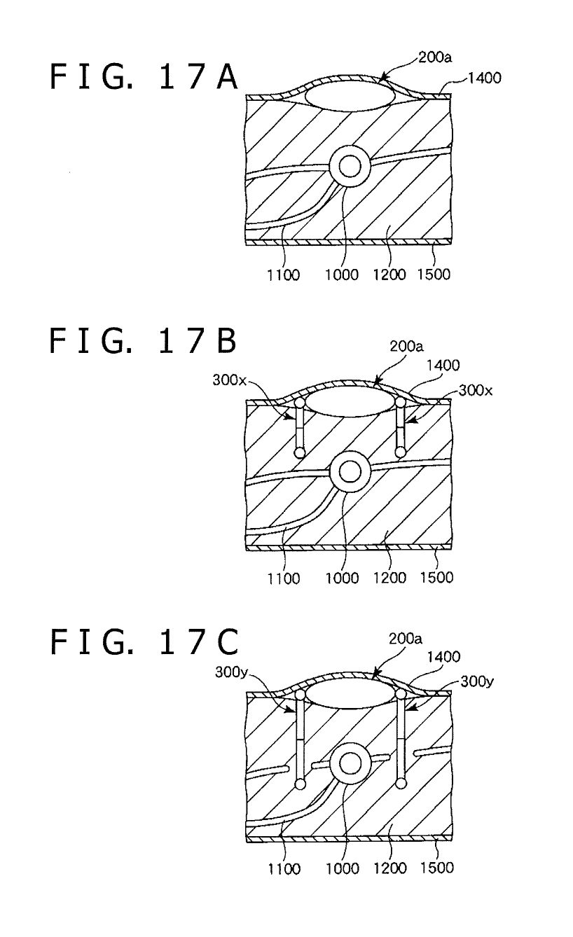

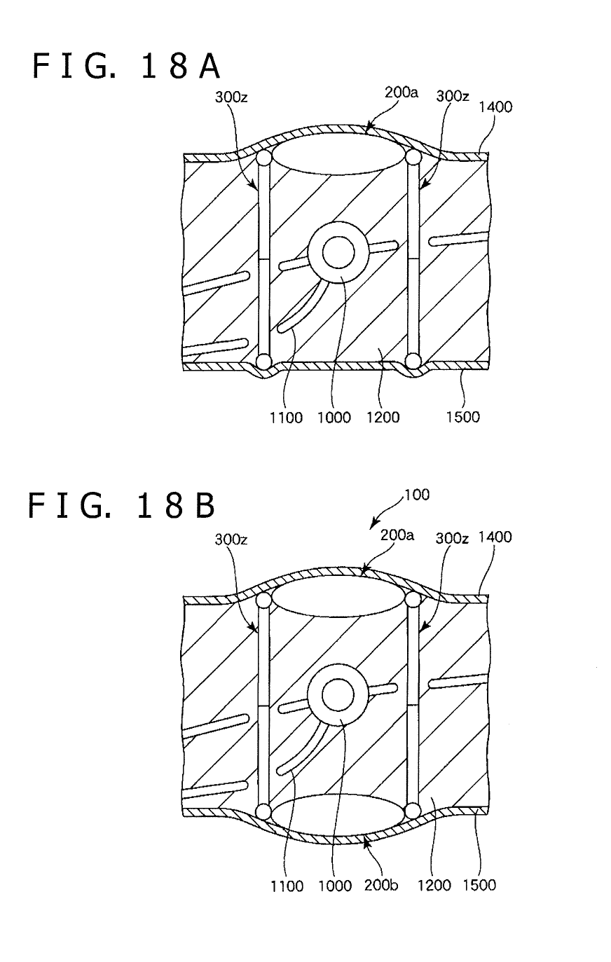

FIG. 16 is a plan view illustrating cutting devices forming a part of a blood vessel dissecting device according to a seventh embodiment of the present disclosure. FIGS. 17A to 18B are views explaining a blood vessel harvesting method carried out using the blood vessel dissecting device that includes the cutting devices shown in FIG. 16.

Referring to these figures, the seventh embodiment will be described below. Features associated with this seventh embodiment that are similar to those in the embodiments described above are identified by common reference numbers. The following detailed description primarily focuses on differences between this seventh embodiment and the embodiments described above, and a detailed description of features already described above will not be repeated.

This seventh embodiment is the same as the aforementioned embodiments, except for differences in the configuration of cutting devices.

As shown in FIG. 16, a blood vessel dissecting device 100 in this embodiment has a cutting device set 30 including three cutting devices 300x, 300y and 300z. In addition, the blood vessel dissecting device 100 in this embodiment has two such cutting device sets 30.

The cutting devices 300x, 300y and 300z are elongated plate-like in shape, and are configured in the same manner, except for differing in width.

Specifically, each of the cutting devices 300x, 300y and 300z includes a tapered blood vessel guide groove section 331, and a blood vessel treating groove section 332 which is located on the proximal side of the blood vessel guide groove section 331 and has a straight shape with a substantially constant width. In addition, each of the cutting devices 300x, 300y and 300z is provided at its distal portion with cutting edge sections 370 provided along the blood vessel guide groove section 331. The blood vessel treating groove section 332 of each of the cutting devices 300x, 300y and 300z is provided with a treating section 340.

In a first step, first, the dissecting device 200a is inserted via an incision, as illustrated in FIG. 17A. FIG. 5A illustrates an example of an incision 1300.

Next, as shown in FIG. 17B, the two cutting devices 300x are connected to the dissecting device 200a, and are inserted into the living body while guiding the cutting devices 300x with the dissecting device 200a. Subsequently, the two cutting devices 300x are drawn out, then, as depicted in FIG. 17C, the two cutting devices 300y are connected to the dissecting device 200a, and are inserted into the living body while guiding the cutting devices 300y with the dissecting device 200a. Next, the two cutting devices 300y are drawn out, then, as illustrated in FIG. 18A, the two cutting devices 300z are connected to the dissecting device 200a, and are inserted into the living body while guiding the cutting devices 300z with the dissecting device 200a. Then, as shown in FIG. 18B, the dissecting device 200b is connected to the two cutting devices 300z, and is inserted into the living body while being guided by the two cutting devices 300z.

Thus, the cutting devices 300x, 300y and 300z differing in width are sequentially inserted into the living body in the order of increasing width. By this, the blood vessel dissecting operation can be carried out more smoothly, as compared with the case where the blood vessel dissecting operation is performed at a time by use of only the cutting device 300z having the large width.

By the seventh embodiment as above, also, the same or equivalent effects to those of the aforementioned embodiments can be produced.

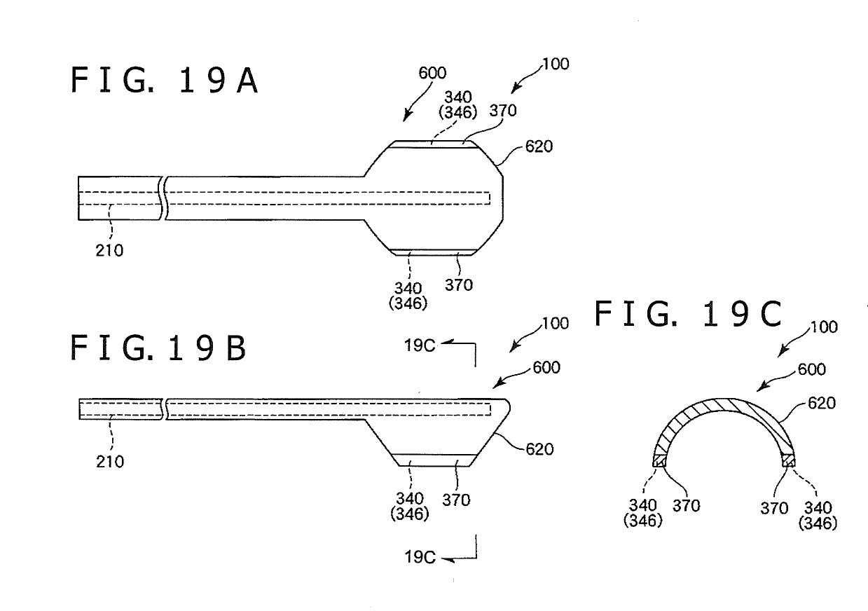

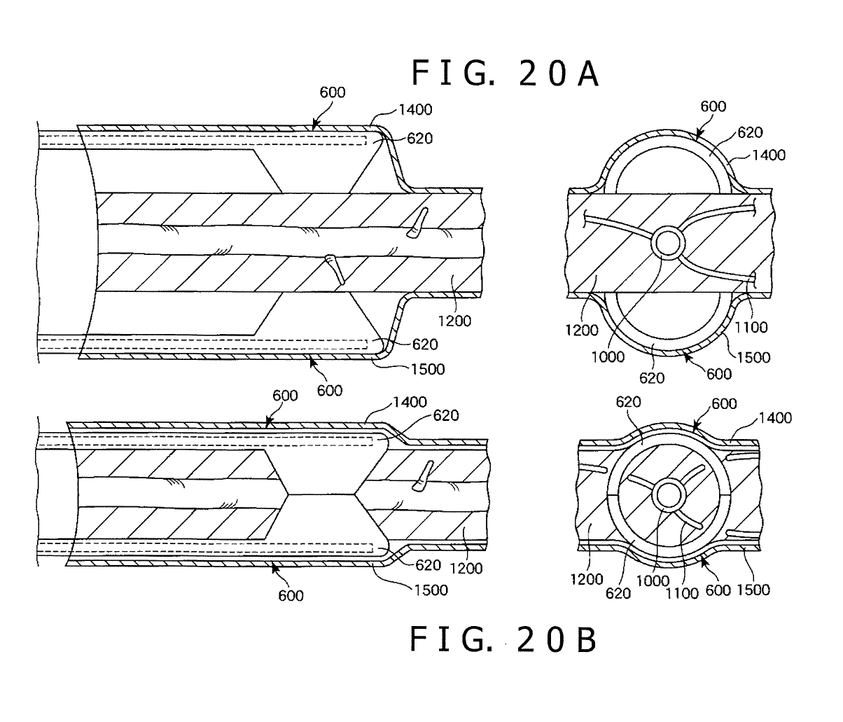

FIGS. 19A to 19C illustrate a dissecting and cutting device forming a part of a blood vessel dissecting device according to an eighth embodiment of the present disclosure, wherein FIG. 19A is a plan view, FIG. 19B is a side view, and FIG. 19C is a sectional view taken along line 19C-19C of FIG. 19B. FIGS. 20A and 20B are views for explaining a blood vessel harvesting method carried out using the blood vessel dissecting device including the dissecting and cutting device shown in FIGS. 19A to 19C.

Note that in the following, for convenience of explanation, the upper side in FIG. 19B will be referred to as “upper side,” the lower side in FIG. 19B will be referred to as “lower side,” the right side in FIG. 19B will be referred to as “forward side” or “distal side (or end),” and the left side in FIG. 19B will be referred to as “rearward side” or “proximal side (or end).”

Referring to these figures, the eighth embodiment will be described below. Features associated with this eight embodiment that are similar to those in the embodiments described above are identified by common reference numbers. The following detailed description will primarily focus on differences between this eighth embodiment and the embodiments described above, and a detailed description of features already described above will not be repeated.

This eighth embodiment is the same as the embodiments described above, except for having a dissecting and cutting device in which a dissecting device and a cutting device are united with each other.

A blood vessel dissecting device 100 in this embodiment includes a dissecting and cutting device 600 as illustrated in FIGS. 19A to 19C. In addition, the blood vessel dissecting device 100 in this embodiment includes two such dissecting and cutting devices 600.

The dissecting and cutting device 600 is elongated in overall shape, and is provided at its distal portion with a dissecting and cutting section 620 having an arched shape projecting while curving from both ends toward outer sides (toward the lower side in FIG. 19B). The distal end of the dissecting and cutting section 620 has a rounded distal surface, so as to function as a dissecting section. In addition, the dissecting and cutting section 620 is provided at both lower end portions thereof with cutting edge sections (cutting sections) 370 for cutting the fat surrounding a great saphenous vein 1000. Further, both lower end portions of the dissecting and cutting section 620 are provided with respective treating sections 340 for cutting and stanching branch vessels. Each treating section 340 has an electrode 346.

As shown in FIG. 19A, the dissecting and cutting device 600 is provided therein with an insertion hole 210 which opens at the proximal end and extends to a distal portion (the dissecting and cutting section 620). Into the insertion hole 210 is inserted an imaging device 400.

In a first step, as illustrated in FIG. 20A, the two dissecting and cutting devices 600 are inserted through an incision 1300 (shown in FIG. 5A) into the living body along a great saphenous vein 1000 while keeping the dissecting and cutting devices 600 spaced from the great saphenous vein 1000. Then, one of the dissecting and cutting devices 600 is disposed on the upper side (the skin 1400 side) of the great saphenous vein 1000, whereas the other of the dissecting and cutting devices 600 is disposed on the lower side (the fascia 1500 side (the bone side)) of the great saphenous vein 1000. In this instance, the two dissecting and cutting devices 600 are so disposed that inner peripheral surfaces of their dissecting and cutting sections 620 are oriented toward (face toward) the great saphenous vein 1000 side. In this operation, one of the dissecting and cutting devices 600 is inserted between fat 1200 and a skin 1400, thereby dissecting the fat 1200 and the skin 1400 from each other. Similarly, the other of the dissecting and cutting devices 600 is inserted between the fat 1200 and a fascia 1500, thereby dissecting the fat 1200 and the fascia 1500 from each other.

In the above-mentioned way, as shown in FIG. 20A, the two dissecting and cutting devices 600 are disposed in facing relation to each other, with the great saphenous vein 1000 interposed between the two dissecting and cutting devices 600.

Next, as shown in FIG. 20B, the two dissecting and cutting devices 600 are moved closer to each other, and lower end portions of their dissecting and cutting sections 620 are mated with each other. In this instance, the two dissecting and cutting devices 600 cut the fat 1200 present on lateral sides of the great saphenous vein 1000 to the left and right with their cutting edge sections 370. Further, when the lower end portions of the dissecting and cutting sections 620 of the two dissecting and cutting devices 600 come close to each other, the two dissecting and cutting devices 600 generate an electric field between the electrodes 346 possessed by the treating sections 340, thereby cutting and stanching the branch vessels 1100.

Then, the two dissecting and cutting devices 600 are moved away from each other. Thereafter, the operation of dissecting the skin 1400 from the fat 1200 and dissecting the fascia 1500 from the fat 1200 as depicted in FIG. 20A and the operation of cutting and stanching the branch vessels 1100 while cutting the fat 1200 as depicted in FIG. 20B are repeated. By these operations, the great saphenous vein 1000 can be dissected in a state in which the great saphenous vein 1000 is covered with the surrounding fat 1200.

By the eighth embodiment as above, also, the same or equivalent effects to those of the aforementioned first embodiment can be produced.

The length of the treating section 340 may, at maximum, be comparable to the length of the blood vessel to be harvested. When the length of the treating section 340 is large, the current passing area would be enlarged, possibly lowering the efficiency of coagulation and cutting. In such a situation, the current passing part may be set to be variable, and current passing may be conducted selectively and sequentially, whereby the lowering in the efficiency due to the increase in the current passing area can be prevented.

While the blood vessel dissecting device, the blood vessel dissecting method and the blood vessel harvesting method according to the described aspects of the present disclosure have been described above on the basis of the embodiments illustrated in the drawings, the disclosure is not limited to the embodiments. The configuration of each component can be replaced by any configuration that has a function equivalent to the original. Any other structure may be added to the configuration according to the present disclosure. In addition, the embodiments may be combined in a desired manner.

While the great saphenous vein is dissected in the state of being covered with fat over the entire perimeter of the vein in the aforementioned embodiments, the great saphenous vein may not necessarily be covered with fat over the entire perimeter thereof. Thus, the great saphenous vein may be dissected in a state where the periphery thereof is partly covered with fat, or in a state of being not covered with fat.

While the case of harvesting a bypass vessel for use in vascular bypass grafting has been described in the aforementioned embodiments, the use of the harvested blood vessel is not limited to the bypass vessel.

The detailed description above describes a blood vessel dissecting device, a blood vessel dissecting method and a blood vessel harvesting method according to various embodiments representing examples of the inventive blood vessel dissecting device, blood vessel dissecting method and blood vessel harvesting method disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A blood vessel dissecting device comprising:
an elongated guide configured to be inserted into a living body along a blood vessel;
a cutting device connectable to the guide, the cutting device being configured to be inserted into the living body and moved in the living body while connected to the elongated guide so that the cutting device is guided by the guide and cuts tissue surrounding the blood vessel in a direction of alignment with the blood vessel; and
the cutting device being deformable by an external force applied to the cutting device while the cutting device is in the living body.

2. The blood vessel dissecting device according to claim 1,
wherein the elongated guide is a first elongated guide, and further comprising a second elongated guide, the first and second elongated guides being positionable in the living body in facing relation to each other and oriented in a direction in which the two guides are aligned with each other, with the blood vessel interposed between the first and second elongated guides, and
the cutting device is connectable to both of the first and second guides.

3. The blood vessel dissecting device according to claim 2, wherein the cutting device is deformable in a direction in which the two guides are aligned, in accordance with a separated distance between the two guides.

4. The blood vessel dissecting device according to claim 3, wherein the cutting device is deformable so as to protrude toward an outer side of a region between the two guides.

5. The blood vessel dissecting device according to claim 1, wherein the cutting device has a bendable/curvable section which is bent or curved by the external force.

6. The blood vessel dissecting device according to claim 1,
wherein the cutting device includes:
a first portion; and
a second portion slidable relative to the first portion in a direction intersecting an insertion direction of the cutting device into the living body.

7. The blood vessel dissecting device according to claim 1, wherein the guide is configured to be inserted between adjacent tissues different in properties so as to dissect the adjacent tissues from each other.

8. The blood vessel dissecting device according to claim 1,
wherein the cutting device includes:
a cutting section configured to cut tissue surrounding the blood vessel; and
a treating section configured to cut and stanch a branch vessel branching from the blood vessel.

9. The blood vessel dissecting device according to claim 1, wherein the elongated guide possesses a flattened shape in transverse cross-section.

10. The blood vessel dissecting device according to claim 1, wherein the cutting device is connectable to the guide by a connecting protuberance that is positionable in a connecting groove, the connecting groove being provided on one of the cutting device and the guide, and the connecting protuberance being provided on the other of the cutting device and the guide.

11. The blood vessel dissecting device according to claim 1, wherein the cutting device includes two movable portions, each movable portion being comprised of at least two links rotationally connected to one another to vary an angle between the two links when the cutting device is deformed.

12. The blood vessel dissecting device according to claim 1, wherein the cutting device includes two movable portions, each movable portion being parallelogram-shaped.

13. The blood vessel dissecting device according to claim 1, wherein the cutting device comprises two movable members each of which includes first and second links, the first link of each movable member possessing a hole in which the second link is positioned, the second link of each movable member axially moving in the hole of the first link when the cutting device is deformed.

14. A blood vessel dissecting method comprising:
inserting a guide into a living body;
moving the guide along a blood vessel in the living body to dissect tissue in a direction of alignment of the blood vessel and the guide;
inserting a cutting device into the living body and moving the cutting device along the blood vessel while guiding the cutting device with the guide;
cutting tissue surrounding the blood vessel in a direction of alignment of the blood vessel and the cutting device, the cutting of the tissue being performed by the cutting device; and
the cutting device being deformed by an external force in the living body during the cutting of the tissue by the cutting device.

15. The blood vessel dissecting method according to claim 14, wherein the cutting device comprises two movable members each of which includes two links that are rotatably connected together so that an angle between the two links can be varied, the external force causing the angle between the two links of each movable member to change.

16. The blood vessel dissecting device according to claim 14, wherein the cutting device comprises two movable members each of which includes first and second links, the first link of each movable member possessing a hole in which the second link is positioned, the method further comprising axially moving the second link of each movable member in the hole of the first link when the cutting device is deformed.

17. The blood vessel dissecting device according to claim 14, further comprising cutting a branch vessel that branches from the blood vessel by contacting the branch vessel with an electrode that is a part of the cutting device and that moves with the cutting device.

18. A blood vessel harvesting method comprising:
inserting a guide into a living body and moving the guide along a blood vessel in the living body to dissect tissue in a direction of alignment of the blood vessel and the guide;
inserting a cutting device into the living body and moving the cutting device along the blood vessel while guiding the cutting device by the guide to cut tissue surrounding the blood vessel in a direction of alignment of the blood vessel and the cutting device;
ligating the blood vessel after dissecting the tissue and after cutting the tissue surrounding the blood vessel, and then cutting the blood vessel;
harvesting the blood vessel, after ligating the blood vessel, so that the blood vessel which is harvested is covered with the tissue; and
during the cutting of the tissue the cutting device is deformed by an external force in the living body.

19. The blood vessel harvesting method according to claim 18, further comprising cutting a branch vessel that branches from the blood vessel while moving the cutting device along the blood vessel by contacting the branch vessel with an electrode that is a part of the cutting device and that moves with the cutting device.

20. The blood vessel harvesting method according to claim 18, wherein:
the inserting of the guide into the living body and moving the guide along the blood vessel includes inserting a first guide into the living body and moving the first guide along the blood vessel to dissect one tissue and inserting a second guide into the living body and moving the second guide along the blood vessel to dissect other tissue; and
the inserting of the cutting device into the living body and moving the cutting device along the blood vessel while guiding the cutting device by the guide includes connecting the cutting device to the first guide and the second guide to guide movement of the cutting device by both the first guide and the second guide.
